(19) United States
(12) Patent Application Publication
(10) Pub. No.: US 2025/0210133 A1
(43) Pub. Date: Jun. 26, 2025

(54) Method Determining the Difference Between the Biological Age and the Chronological Age of a Subject

(71) Applicant: Genknowme S.A., Lausanne (CH)

(72) Inventors: Sébastien Nusslé, Lausanne (CH); Semira Gonseth Nusslé, Lausanne (CH)

(21) Appl. No.: 18/847,544

(22) PCT Filed: Mar. 15, 2023
(86) PCT No.: PCT/EP2023/056637

§ 371 Date: Sep. 16, 2024

(30) Foreign Application Priority Data
Mar. 15, 2022 (EP) 22162216.0

(51) Int. Cl. G16B 20/40; G16B 20/20; G16B 50/30

(52) U.S. Cl. CPC G16B 20/40; G16B 20/20; G16B 50/30

(57) ABSTRACT

The method determines the difference between the biological age and the chronological age in connection with the influence of measurable lifestyle factors, wherein the method comprises the steps of: providing a biological age database of a reference population comprising methylation levels of a plurality of predetermined methylation sites of human cells related to the biological age for the members of said reference population. A similar life style related database of said same reference population is provided. For a plurality of sets of combinations of methylation sites selected from the related database the biological age for each member of the reference population is calculated and for each of the sets combinations, two maximum are calculated: the prediction of chronological age for said members with general linear models and the proportion of variability in the difference between biological age and chronological age with the biological age values calculated as mentioned above with conditional (Bayesian) statistics. Then the set with the maximized combined selection value is chosen and the parameters of said set are used together with the methylation levels of a subject U to determine the difference between the calculated biological age and the chronological age of said subject wherein said difference results in the youth capital.

### CROSS-REFERENCE TO RELATED APPLICATIONS

This application is the United States national phase of International Patent Application No. PCT/EP2023/056637 filed Mar. 15, 2023, and claims priority to European Patent Application No. 22 162 216.0 filed Mar. 15, 2022, the disclosures of which are hereby incorporated by reference in their entireties.

### BACKGROUND OF INVENTION

### Technical Field

The present invention relates to a method determining the difference between the biological age and the chronological age of a subject.

### Description of Related Art

WO 2018/229032 A1 relates to a method for determining the biological age of human skin comprising providing human skin cells, determining a methylation level of at least two CpG-dinucleotides of a specific region of at least one chromosome of said skin cells and determining the biological age of said skin cells by comparing said determined methylation level with empirically determined data representing a correlation between the methylation level of the CpG-nucleotide and the chronological age of at least one human individual.

The challenge of chronic disease is immense, and health promotion, preventative care and disease prevention programs could dramatically decrease this burden. The increased understanding of lifestyle-related illness has resulted in an unprecedented intensification in health consciousness, and personalized interventions and effective communication are key elements for such programs. The challenge being how to formulate health risk information in a way that can be understood and that motivates behavior change. What is lacking is a simple metric to objectify the overall lifestyle status, i.e., the relationship between lifestyle habits and health outcomes. It is common knowledge that good nutrition is important, but currently no one can objectively quantify the real effect of their personal lifestyle on their own health. Epigenetics provides this missing link by measuring, within the DNA, the actual effects of lifestyle and lifestyle changes on biological age, a simple and easy to understand indicator.

Drastic changes occur in DNA methylation patterns with aging. For instance, more than 20,000 epigenetic biomarkers have been found to be associated with aging in over 20 studies encompassing thousands of participants in a meta-analysis of epigenome-wide epigenetic (DNA methylation) associations studies (EWASes) mostly of white blood cells (available in the EWAS atlas, at http://bigd.big.ac.cn/ewas); the most robust biomarkers were located in the genes NHLRC1, EDARADD, SCGN, and FHL2. DNA methylation biomarkers of aging can be considered as a summary measure of all environmental and lifestyle influences on DNA methylation, and some of these biomarkers were associated with various health outcomes such as cardiovascular disease, Alzheimer's disease, body mass index, and longevity (all reviewed in Andersen, et al.)

References are mentioned at the end of the prior art section of the specification.

Tobacco smoking and alcohol drinking are two major risk factors for cardiometabolic diseases. They both produce numerous epigenetic modifications. Indeed, meta-analyses of tens of thousands of participants have reported strong and reproducible associations between DNA methylation levels at specific loci throughout the epigenome with both tobacco smoking and alcohol intake. In particular, Joehanes, et al. have conducted a meta-analysis of epigenome-wide DNA methylation associations studies (EWASes), estimating associations between smoking status and DNA methylation of peripheral white blood cells. This analysis included 15,907 participants in 16 cohorts (including 2,433 current, 6,518 former, and 6,956 never smokers). More than 2,600 CpG sites (annotated to more than 1,400 genes) had different methylation levels in function of tobacco smoking status. Among the top most associated CpGs sites, were sites annotated to HIVEP3, SGIP1, SKI or AHRR genes. In addition, it was demonstrated in a clinical trial on smoking cessation that one CpG site annotated to the AHRR gene (a site recurrently identified from EWASes of tobacco smoking) showed different DNA methylation levels before and 6 months after the participants successfully quit smoking. Regarding alcohol intake, Liu, et al. meta-analyzed white blood cell-based EWASes of alcohol intake in more than 13,000 participants from 13 cohorts. They identified over 300 CpG sites significantly associated with alcohol intake. Stratifying participants by ethnicity, they developed an epigenetic signature able to discriminate heavy versus light/no drinking using 5 CpG sites with a good precision.

Over 700 DNA methylation markers were associated in the diet regarding fruits and juices consumption in 2,148 participants of the Framingham study. A pathway analysis revealed that these markers were located in immune response and telomere regulation pathways.

About a dozen DNA methylation markers were significantly associated with total physical activity, and leisure-time physical activity in 1,242 participants of the Melbourne Collaborative Cohort Study. The most associated marker was annotated to the SAA2 gene, which is involved in cardiovascular disease development. The association of this particular marker with physical activity levels has been replicated in an independent study in post-menopausal women.

However, as previously demonstrated, inter-individual DNA methylation variation at CpG sites sensitive to environmental exposures such as smoking can be confounded by inter-individual genetic variation of Single-Nucleotide-Polymorphisms (SNPs). Indeed, about 25% of the DNA methylation variation in the human genome is influenced by genetic variation of SNPs located in proximity. Such SNPs are called methyl-quantitative trait loci (methyl-QTL) and they modify DNA methylation levels by modulating the binding abilities of transcription factors.

Such genetic confounding can therefore potentially decrease the accuracy of any DNA methylation-based-only predictive signature of exposures, and genetic variation must be accounted for in developing such instruments.

WO 2019/143845 is related to biomarkers for life expectancy and morbidity based on phenotypic age and DNA methylation.

WO 2020/076983 A1 discloses DNA methylation based biomarkers for life expectancy and morbidity. The predictor of lifespan, DNAm GrimAge is a composite biomarker based inter alia on lifestyle factors and smoking pack-years. The technique used to create the GrimAge DNAm-based estimator differs from previous estimators in that a two-step approach is used to create the final estimator. Namely, in a first step, DNAm-based biomarkers are identified that served as surrogates for tobacco exposure (smoking pack-years), as well as various plasma proteins evidenced to be associated with mortality or morbidity. Then, in the second step, time-to-death was regressed on the previously identified surrogate DNAm-based biomarkers, chronological age, and sex using an elastic net model to identify the most important predictors for predicting time-to-death; this resulted in a final selection of 10 predictors; the linear combination of which equates to the estimated logarithm of the hazard ratio for mortality. Through linear transformation of the estimate, inventors created an age estimate (DNAm GrimAge) that maximizes for an association with time-to-death, thereby allowing for a DNAm-based age estimator that demonstrates superior performance in estimating risk of all-cause mortality and risk of coronary heart disease.

### SUMMARY OF THE INVENTION

Based on this prior art it is an object of the present invention to provide a method determining the difference between the biological age and the chronological age in connection with the influence of measurable lifestyle factors. The measurable lifestyle factors are measurable epigenetic markers. The difference between the biological age and the chronological age is also called youth capital.

This object is achieved with the teaching as described herein.

Each set Sₘₛ can comprise a combination between 10 to 50 methylation sites. The more database entries are present for different methylation sites, the more options are open for the combinations. The number p of sets used in c.) and d.) of the method as combinations from the n^{ls}ₘₛ possible methylation sites can be chosen as p equal to the binomial coefficient “n^{ls}ₘₛ choose k”, wherein n^{ls}ₘₛ>=k>=10 which can be a number of p in millions.

The methylation sites of human cells related to life style factors can comprise one or more methylation sites of the Tobacco smoking epigenetic signature, of the Alcohol drinking epigenetic signature, of the Fruits & vegetables consumption epigenetic signature, and/or of the Exercise epigenetic signature in said biological sample of a reference population. Although it is possible to choose only methylation sites of one epigenetic signature, a greater number of methylation sites for each of the epigenetic signature provides better maximization.

The methylation sites related to the life style factors can be selected from the list of Table 1 marked life style factor methylation sites. The methylation sites related to the biological age can be selected from the list of Table 1 marked biological age methylation sites. Table 1 is an example based on built databases relating to specific factor related methylation sites.

The database entries for methylation sites can providing methylation levels of the plurality of methylation sites of human cells related to life style factors for the same reference population and for the subject U comprises detecting the typology of at least one SNP affecting methylation site selected from the list in Table 2 and using the value under “CpG Value for allele combination” as multiplier for the associated CpG.

The human cells used to determine methylation levels for specific methylation sites can be solid tissue, blood, fecal or saliva sample that comprises genomic DNA. The determination of methylation levels for specific methylation sites of the reference population can be done on the same or different types of human cells.

The methylation value of a CpG site in a population of human cells can be the average degree of methylation of said CpG methylation site in a population of a sample of cells, usually comprising hundreds up to and over hundreds of thousands of cells.

Furthermore, the typology of at least one SNP affecting methylation site can be multiplied with the values of the detected methylation levels and associated SNP into a score for each lifestyle factor, wherein each methylation site value is weighted evenly or differently in reaching the score(s). This can be done by two methods, the weighted one is the regression that can be apply, and the evenly one is taking the average methylation in a region:

After having determined the youth capital of a subject, the method according to the invention can be repeated in time and subsequently determining the change in difference between the chronological age and the biological age between the first and the second point in time to evaluate the possible success after a change in alcohol consumption, exercise, tobacco consumption, nutrition.

Further embodiments of the invention are laid down in the dependent claims.

### REFERENCES

Hannum, G. et al. Genome-wide Methylation Profiles Reveal Quantitative Views of Human Aging Rates. Mol. Cell 49, 359-367 (2013).
Horvath, S. DNA methylation age of human tissues and cell types. Genome Biol. 14, 3156 (2013).
Chen, B. H. et al. DNA methylation-based measures of biological age: meta-analysis predicting time to death. Aging 8, 1844-1859 (2016).
Lu, A. T. et al. DNA methylation GrimAge strongly predicts lifespan and healthspan. Aging 11, 303-327 (2019).
Brock C. Christensen, Karl T. Kelsey & Carmen J. Marsit. Influence of Environmental Factors on the Epigenome. in Epigenetic Epidemiology (2012).
Andersen, A. M., Dogan, M. V., Beach, S. R. & Philibert, R. A. Current and future prospects for epigenetic biomarkers of substance use disorders. Genes 6, 991-1022 (2015).
Joehanes, R. et al. Epigenetic Signatures of Cigarette SmokingCLINICAL PERSPECTIVE. Circ. Cardiovasc. Genet. 9, 436-447 (2016).
Liu, C. et al. A DNA methylation biomarker of alcohol consumption. Mol. Psychiatry 23, 422-433 (2018).
Nicodemus-Johnson, J. & Sinnott, R. A. Fruit and Juice Epigenetic Signatures Are Associated with Independent Immunoregulatory Pathways. Nutrients 9, (2017).
Van Roekel, E. H. et al. Physical Activity, Television Viewing Time, and DNA Methylation in Peripheral Blood. Med. Sci. Sports Exerc. 51, 490-498 (2019).
Gonseth, S. et al. Genetic contribution to variation in DNA methylation at maternal smoking-sensitive loci in exposed neonates. Epigenetics 1-10 (2016) doi: 10.1080/15592294.2016.1209614.
Tsaprouni, L. G. et al. Cigarette smoking reduces DNA methylation levels at multiple genomic loci but the effect is partially reversible upon cessation. Epigenetics 9, 1382-1396 (2014).
Kent, W. J. et al. The Human Genome Browser at UCSC. Genome Res. 12, 996-1006 (2002).

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows a diagram of explained variance when the method according to an embodiment of the invention is applied with a SKIPOGH reference population;

FIG. 2 shows two diagrams with a distribution of reference population categorized by smoking status for a method according to an embodiment of the invention;

FIG. 3 shows two diagrams with a distribution of reference population categorized by drinking status for a method according to an embodiment of the invention;

FIG. 4 shows a diagram between the chronological epigenetic age in the SKIPOGH reference population, and

FIG. 5 shows a flowchart of the main steps of the method according to an embodiment of the invention.

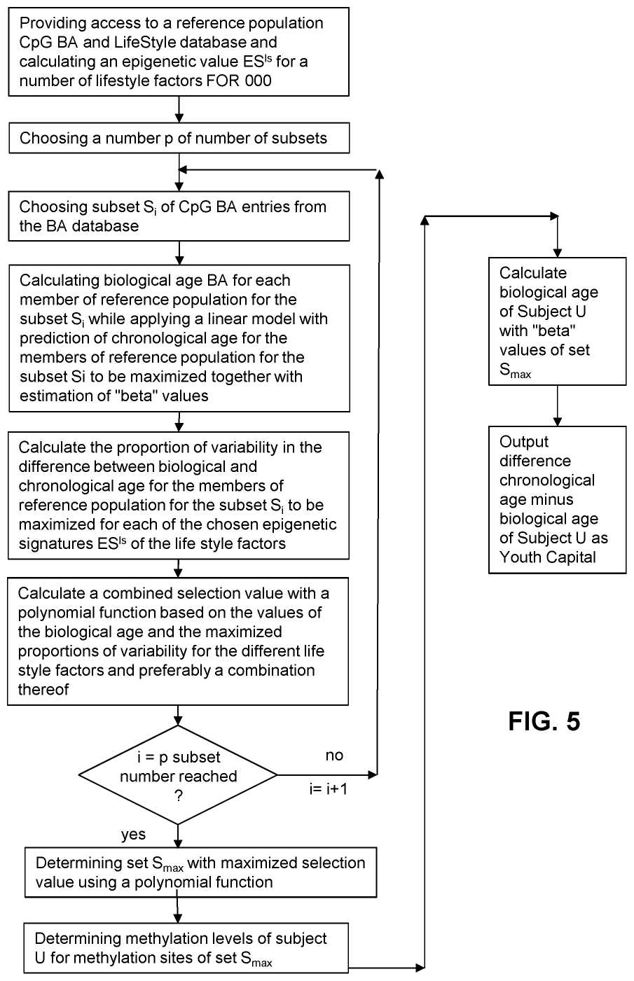

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

Reference throughout this specification to “one aspect”, “an aspect”, “another aspect”, “a particular aspect”, “combinations thereof” means that a particular feature, structure or characteristic described in connection with the invention aspect is included in at least one aspect of the present invention. Thus, the appearances of the foregoing phrases in various places throughout this specification are not necessarily all referring to the same aspect. Furthermore, the particular features, structures, or characteristics may be combined in any suitable manner in one or more aspects.

The term “comprise/comprising” is generally used in the sense of include/including, that is to say permitting the presence of one or more features or components. The terms “comprise(s)” and “comprising” also encompass the more restricted ones “consist(s)” and “consisting”, respectively.

As used in the specification and claims, the singular form “a”, “an” and “the” include plural references unless the context clearly dictates otherwise.

As used herein, “at least one” means “one or more”, “two or more”, “three or more”, example . . . etc. For example, at least one SNP, means e.g. a combination of two, three, four, five, six, etc. . . . SNPs.

The term “about”, particularly in reference to a given quantity or percentage, is meant to encompass deviations of plus or minus ten (10) percent (+/−10%).

As used herein the terms “subject” or “patient” are well-recognized in the art, and, are used for human beings. In some cases, the subject is a subject in need of treatment or a subject with a disease or disorder. However, in other aspects, the subject can be a normal subject. The term does not denote a particular age or sex. Thus, adult and newborn subjects, whether male or female, are intended to be covered.

Within the present specification a number of variables are used to specify features:
N_{BA}=number of members of the reference population
n^{ba}ₘₛ=number of methylation sites in the biological age database
n^{ls}ₘₛ=number of methylation sites in the life style related database
p=number of chosen sets of combinations
S^{ba}ₘₛ=set of methylation sites selected from the biological age related database
S^{ls}ₘₛ=set of methylation sites selected from the life style related database
m^{ba}ₚ=number of methylation sites selected from the biological age related database<=n^{ba}ₘₛ
m^{ls}ₚ=number of methylation sites selected from the life style related database<=n^{ls}ₘₛ, wherein
Is is any of the life style factors or a combination of the life style factors. (CpG(1) . . . . CpG(n^{ba/ls}ₘₛ))=methylation sites of the database (either biological age of life style related)
(CpG(i,1), . . . , CpG(i,n^{ba/ls}ₘₛ)=methylation level of CpG sites for the i-th member of the reference population (either biological age of life style related)
beta₀ and beta with I taking predetermined values I ∈ {1, 2, . . . , m^{ba/ls}ₚ} of the determined set Sₘₐₓ;

As used herein, the “youth capital” refers to the difference between biological and chronological age, which is linked to external factors such as tobacco and alcohol consumption, diet adequacy, and physical activity.

A “reference population” or “cohort” as used herein refers to sample of a larger population in which participants have been randomly sampled from population registries. It is hypothesized that the reference population is a representative sample of a population and therefore seeks to accurately reflect the characteristics of the larger population. The larger population can be understood as an ethnicity, for example Caucasians, a sub-ethnicity such as Slavic people, a country with several ethnicities, for example Chinese people, a region, or even a continent, the African or South American population for example. Examples of a reference population or cohort comprise the Swiss Kidney Project on Genes in Hypertension study (SKIPOGH). The database accessed in the present examples of the invention is a database comprising for each member of the reference population:
methylation level of CpG sites related to biological age,
methylation level of CpG sites related to the mentioned life style factors,
the chronological age of the member at the time of taking the samples.

The database is in the present description sometimes divided into a database comprising the methylation levels of CpG sites related to biological age on one side and the methylation levels of CpG sites related to the mentioned life style factors on the other side. The chronological age of the member(s) at the time of taking the samples to evaluate the methylation level values is then usually stored in one or both of these databases.

The term “methylation site” as used herein refers to a CpG position that is potentially methylated. Methylation typically occurs in a CpG containing nucleic acid. The CpG containing nucleic acid may be present in, e.g., in a CpG island, a CpG doublet, a promoter, an intron, or an exon of gene.

Hyper or hypo-methylation of the methylation sites (e.g., methylation status) can be assessed by detecting methylation status and comparing a value to a relevant reference level. For example, the methylation status of one or more markers can be indicated as a value. The value can be one or more numerical values resulting from the assaying of one or more biological sample(s), and can be derived, e.g., by measuring methylation status of the marker(s) in the sample(s) by an assay, or from a dataset obtained from a provider such as a laboratory, or from a dataset stored on a server. DNA methylation of the methylation markers (or markers close to them) can be measured using various approaches, which range from commercial array platforms (e.g. from Illumina™) to sequencing approaches of individual genes. This includes standard lab techniques or array platforms. A variety of methods for detecting methylation status or patterns have been described in, for example U.S. Pat. Nos. 6,214,556, 5,786,146, 6,017,704, 6,265,171, 6,200,756, 6,251,594, 5,912,147, 6,331,393, 6,605,432, and 6,300,071 and US Patent Application publication Nos. 20030148327, 20030148326, 20030143606, 20030082609 and 20050009059, each of which are incorporated herein by reference. Other array-based methods of methylation analysis are disclosed in U.S. patent application No. 20050196792. For a review of some methylation detection methods, see, Oakeley, E. J., Pharmacology & Therapeutics 84:389-400 (1999). DNA methylation was determined using the Illumina Infinium MethylationEPIC BeadChip

In certain aspects of the invention measuring methylation status comprises, performing methylation specific PCR (MSP), real-time methylation specific PCR, methylation-sensitive single-strand conformation analysis (MS-SSCA), quantitative methylation specific PCR (QMSP), PCR using a methylated DNA-specific binding protein, high resolution melting analysis (HRM), methylation-sensitive single-nucleotide primer extension (MS-SnuPE), base-specific cleavage/MALDI-TOF, PCR, real-time PCR, Combined Bisulfite Restriction Analysis (COBRA), methylated DNA, immunoprecipitation (MeDIP), a microarray-based method, pyrosequencing, or bisulfite sequencing.

Usually, the methylation status will be expressed as a beta-value, i.e., the percentage of methylated DNA string at a given location.

Preferably, the method comprises the detection of the methylation status as values of a plurality of methylation sites related to lifestyle factors selected from the list of a table as e.g. Table 1 and a plurality of methylation sites related to the biological age selected from the list of a table as e.g. Table 1.

As used herein, lifestyle factors refer to external factors such as tobacco and alcohol consumption, diet adequacy, and physical activity. In the present case, the lifestyle factors are selected among the group comprising tobacco smoking, alcohol drinking, fruits & vegetables consumption and/or exercise.

The “biological age” refers to a measure of ageing that is more related to longevity and the risk of chronic diseases than chronological age. It accounts for the effect of lifestyle, either deterioration due to unhealthy habits or protection due to healthy habits whereas the “chronological age” refers to the amount of time that has passed from the birth of a subject to the given date.

The epigenetic biological age could be defined with three properties:
1) Biological aging results as an unintended consequence of both developmental programs and maintenance program, the molecular footprints of which give rise to epigenetic markers.
2) The precise mechanisms linking the innate molecular processes to the decline in tissue function probably relate to both intracellular changes (leading to a loss of cellular identity) and subtle changes in cell composition, for example, fully functioning somatic stem cells.
3) At the molecular level, biological age is a proximal readout of a collection of innate aging processes that conspire with other, independent root causes of ageing to the detriment of tissue function.

As used herein, a “biological sample” refers to a sample of tissue or fluid isolated from a subject, including but not limited to, for example, urine, blood, plasma, serum, fecal matter, bone marrow, bile, spinal fluid, lymph fluid, samples of the skin, external secretions of the skin, respiratory, intestinal, and genitourinary tracts, tears, saliva, milk, blood cells, organs, biopsies, and also samples containing cells or tissues derived from the subject and grown in culture, and in vitro cell culture constituents, including but not limited to, conditioned media resulting from the growth of cells and tissues in culture, recombinant cells, stem cells, and cell components. In preferred aspects, the biological sample is a tissue e.g. solid tissue, blood, fecal or saliva sample that comprises genomic DNA.

A single-nucleotide polymorphism (SNP) is a substitution of a single nucleotide at a specific position in the genome, that is present in a sufficiently large fraction of the population (e.g. 1% or more). SNPs can influence the methylation of nearby methylation sites, e.g. CpG sites. It will be understood by the skilled artisan that SNPs may be used singly or in combination with other SNPs. Preferably, the SNPs affecting methylation sites are selected from the list of a table as e.g. Table 2.

Typically, the step of detecting the presence of at least one SNP and determining its typology (i.e. which allele is present) comprises amplifying a nucleic acid present in the biological sample. In an aspect, the step of detecting the presence of at least one SNP comprises a technique selected from the non-limiting group comprising, e.g., mass spectroscopy, RT-PCR, microarray hybridization, pyrosequencing, thermal cycle sequencing, capillary array sequencing, solid phase sequencing, a hybridization-based method, an enzymatic-based method, a PCR-based method, a sequencing method, a ssDNA conformational method, and a DNA melting temperature assay.

The method determining the difference between the biological age and the chronological age in connection with the influence of measurable lifestyle factors comprises several steps, explained as follows:

a.) providing access to methylation level values in a biological age database of a reference population comprising n^{ba}ₘₛ methylation levels (CpG(i,1), . . . , CpG(i,n^{ba}ₘₛ) for i=1 to n_{BA} of a plurality of predetermined methylation sites (CpG(1) . . . . CpG(n^{ba}ₘₛ)) of human cells related to the biological age for the n_{BA} members of said reference population as well as their chronological age.

The reference population has n^{ba}ₘₛ members. The database comprises data for n_{BA} predetermined methylation sites.

b.) providing access to methylation level values in a life style related database of said same reference population comprising n^{ls}ₘₛ methylation levels (CpG(i,1) . . . . CpG(i,n^{ls}ₘₛ)) for i=1 to n_{BA} of a plurality of methylation sites (CpG(1) . . . . CpG(n^{ls}ₘₛ)) of human cells related to life style factors for the n_{BA} members of said reference population.

The same reference population with its n^{ba}ₘₛ members are the entries for the life style related database with data for n_{BA} predetermined methylation sites related to life style factors. In fact the database is a combination of a number of 2 dimensional arrays of user entries for different life style factors, which are different one to the other. Interesting life style factors are related to tobacco use, vegetable and fruit consumption, sportive activities, but can also comprise data relating to alcohol consumption or other life style relating values.

The values of methylation level of CpG sites related to the mentioned life style factors in above mentioned databases can be reduced to one to five different values as epigenetic signatures ES^{ls} for each of the lifestyle factors tobacco, alcohol, fruits & vegetables, exercise and a combined value for these four lifestyle factors. This relates to choosing once for each lifestyle factor to be included to generate an ES^{ls} value a set Sₘₛ of combinations of mₚ methylation sites selected from the life style related database with for the set comprising 1 or more different CpG(j) ∈ Sₘₛ with j taking predetermined values j ∈ {1, 2, . . . , mₚ} of the chosen combination.

c.) choosing a plurality of p sets Sₘₛ of combinations of mₚ methylation sites selected from the biological age related database with for each set comprising 1 or more different CpG(j) ∈ Sₘₛ with j taking predetermined values j ∈ {1, 2, . . . , mₚ} of the chosen combination.

The choosing step is in reality to be seen in connection with steps d.) to f.), since the calculations are performed for any chosen set of combinations of methylation sites. First a set of methylation sites is chosen, preferably at least 10, if the database comprises mₚ>=10 methylation sites. Then data relating to these methylation sites if compiled according to step d.) and then another set is chosen until the predetermined number of p sets is reached. The number p has to be chosen a large number, e.g. 10′000 up to more than a 1′000′000, with each time a different combination of the chosen methylation sites, in number and in choice.

Therefore c.) is the aggregate of the steps:
Initially a number p of number of subsets is chosen.
Then for the first subset, the CpG methylation site entries in the database are accessed, i.e. CpG(i,l) with i for the i-th member of the reference population and I for the values I ∈ {1, 2, . . . , m^{ba}ₚ} of the determined set Si.

d.) calculating the biological age BA (i) for each member i of said reference population, i=1 to n_{BA}, for set I out of p sets Sₘₛ of combinations with the formula:

BA (i)=beta₀+Σbetaj×CpG(i,j),

with CpG(i,j) being the methylation level for the member i and methylation site j and betaj being a parameter multiplying the methylation level of the associated CpG(j).

As mentioned above, the biological age for each member of the reference population is calculated as a linear function of a base age beta, and a sum of factors beta; multiplied with the CpG value for this member and the methylation site.

These steps are conducted with conditions:
e.) while for each of the p sets Sₘₛ of combinations
e1) the prediction of chronological age for the members i of the reference population is maximized with general linear models and
e2) the proportion of variability in the difference between biological age and chronological age with the biological age values calculated in d) is maximized with conditional (Bayesian) statistics,
e3) calculating a combined selection value based on the maximized chronological age of
e1) and the variability value of e2) with a polynominal function.

This means that initially the prediction of the chronological age of the members of the reference population is maximized for the subset in question applying a linear model which provides the predicted chronological age based on the biological age CpGs and the “beta” values.

As mentioned above, the database comprises entries for the chronological age of each member of the reference population. Now a value is determined as the maximum value for all chronological ages of the reference population members based on general linear models.

Then the proportion of variability is calculated for each of the above mentioned life style factor values represented by the ES^{ls} for each life style value (i.e. one to four values) and preferably a combination of the four life style values, which means in total five values.

The difference with such a chronological age and the biological age is minimized, i.e. the variability is maximized.

Then, in total there are up to six resulting values. One for the BA value of above e1) and up to five values for e2).

These values are up to six local maximum of intertwined variables and are the input for the third sub step and a combined selection value is determined based on a polynominal function of the up to six values of e1) and e2). A simple combination would be the simple addition of the normalized values of e1) and the up to five values for e2), as having the parameters in the linear equation e.g. 35%, 25%, 20%; 10% and 5% and 5%.

The the loop is closing until all subsets are estimated in the method and a corresponding number of selection values are present leading to the evaluation steps based on the technically transformed information from the CpG values. The next step is determining one specific set Sₘₐₓ having a specific combined selection value as follows:

f.) determining the set Sₘₐₓ out of the p sets Sₘₛ with the maximized combined selection value having the parameters beta, and beta with I taking predetermined values I ∈ {1, 2, . . . , mₚ} of the determined set Sₘₐₓ.

This gives rise to a sequence of beta, and beta values which are then used in connection with the predetermined methylation levels of the subject or user U for which the biological age is to be determined.

g.) providing methylation levels (CpG(U,1) . . . . CpG(U,n^{ls}ₘₛ)) of human cells related to a subject U for all methylation sites related to life style factors chosen in the set Sₘₐₓ,

A number of cells of the subject and user U are used to determine the methylation levels of the methylation sites related to CpG factors chosen in the set Sₘₐₓ. This allows determining the biological age of the subject as in the next step:

h.) determining the biological age of said subject U as

$$BA ( S ) = {beta}_{0} + \sum {beta}_{I} \times CpG ( U , I ) ,$$

with CpG(U,I) being the methylation level for the subject U and methylation site I from the determined set Sₘₐₓ.

Finally as an output step, the difference between the biological age as calculated and the chronological age of said subject U is given as the youth capital.

i.) determining the difference between the biological age calculated in h.) and the chronological age of said subject U wherein said difference results in the youth capital.

The method preferably further comprises a step of combining the values of the detected methylation status as values and associated SNP into a score for each lifestyle factor, wherein each methylation site value is weighted evenly or differently as defined in the summary of the invention in reaching the score(s), wherein providing methylation levels of the plurality of methylation sites of human cells related to life style factors for the same reference population and for the subject U comprises detecting the typology of at least one SNP affecting methylation site selected from the list in Table 2 and using the value under “CpG Value for allele combination” as multiplier for the associated CpG.

In short the above mentioned steps c.) to e.) comprise calculations based on:
1. A large number (several million) of random combinations of selected CpG for biological age is drawn from the databases or as reflected in Table 1.
2. If a random combination comprises one or more CpG is associated with one or more SNP present in Table 2, the SNP is also selected.
3. For each random combination of CpG and SNP selected in the previous steps, a linear model with chronological age as response variable and the combination on CpG and SNP as explanatory variables is performed.
4. For each randomly selected model:
   a. The proportion of variance (R2) explained by the model is calculated as goodness-of-fit measure
   b. Another linear model is performed with the residuals of the model estimates in step 3 as response variable and the lifestyle epigenetic scores as explanatory variables. These are up to five values, i.e. one value for each life style factor and a sum up value for a linear combination of the four life style values.
   c. The proportion of variance (R2) explained by the second model is also calculated as goodness-of-fit measure.
5. The model that maximizes both proportions of variance (steps 4a and 4c) is selected as the final model, wherein the model is selected as a linear combination of the results of step 4a and 4c, i.e. up to six factors with one for biological age, up to four for the different lifestyle factors and the said combination value for the life style factors.

The linear combination can be a combination of thresholds as e.g. the value of 4a being greater than a first threshold and the value of 4c being greater than a second threshold. Additionally each single lifestyle factor can also have its own threshold. Then the maximal value of all of these values can be chosen.

The method of the invention further comprises a step of determining the biological age of said subject with the score determined in d) as follows:

$$Biological Age = {betaBA}_{0} + b {etaBA}_{1} \times CpG ( BA 1 ) + {betaBA}_{2} \times CpG ( BA 2 ) + … {betaBA}_{i} \times CpG ( BAi ) ,$$

with betaBAᵢ being a parameter multiplying the methylation value of the i-th CpG

For example, if the score has only 2 CpGs, CpG(BA1) and CpG(BA2), and if the associated betas (betaBA₀, betaBA₁, betaBA₂) have been estimated to be respectively, 30, 10 and −5, then the biological age of an individual with the respective CpG values of 0.6 and 0.2, would be: Biological Age=30+10*0.6-5*0.2=35 years old.

The difference between the biological age calculated in d) score and the chronological age of said subject results in the youth capital.

A method for determining the youth capital of a subject, the method comprising the steps of
a) detecting, in a biological sample of said subject,
a1) the methylation status of a plurality of methylation sites related to lifestyle factors selected from the list of Table 1 consisting of the Tobacco smoking epigenetic signature, the Alcohol drinking epigenetic signature, the Fruits & vegetables consumption epigenetic signature, and/or the Exercise epigenetic signature,
a2) the methylation status of a plurality of methylation sites related to the biological age selected from the list of Table 1,
b) detecting the typology of at least one SNP affecting methylation sites selected from the list in Table 2,
c) combining the values of the detected methylation status and associated SNP into a score for each lifestyle factor, wherein each methylation site value is weighted evenly or differently in reaching the score(s),
d) determining the biological age of said subject with the score determined in d)
e) determining the difference between the biological age score obtained in d) and the chronological age of said subject wherein said difference results in the youth capital.

An example of a Lifestyle factor Score is LFS=beta0+beta (1)×CpG(1)+beta (2)×CpG(2)+ . . . beta (i)×CpG(i) which can be applied for each of the individual life style factors as shown below. These calculations are examples for a specific result set, where the variables are running from 1 to e.g. 5 for Tobacco, i.e. that they are renumbered and not representative for the first up to fifth CpG of the tobacco lifestyle factor entries of e.g. Table 1.

### Tobacco Score (TS):

beta0 is comprised between about −1.6 and about −0.6
beta (1) is comprised between about −2.6 and about −1.6
beta (2) is comprised between about −5.0 and about −4.0
beta (3) is comprised between about 0.3 and about 1.1
beta (4) is comprised between about 2.2 and about 3.5
beta (5) is comprised between about 1.0 and about 1.8

An example of a Tobacco Score is TS=−1.0−2.1×cg05575921−4.4×cg26703534+0.7×cg23480021+2.9×cg08118908+1.4×cg00336149

Alcohol Score (AS):
beta0 is comprised between about 35 and about 50
beta (1) is comprised between about −13 and about −10
beta (2) is comprised between about −4.5 and about −6
beta (3) is comprised between about 5 and about 7
beta (4) is comprised between about 7.5 and about 9

An example of an Alcohol Score is AS=44.7-3.8×cg12873476-11.2×cg06690548-5.3×cg20970369+6.1×cg03497652+8.3×cg26248486

### Physical Activity Score (PAS):

beta0 is comprised between about 7 and about 10
beta (1) is comprised between about 1.5 and about 3
beta (2) is comprised between about −3 and about −1.7

An example of a Physical activity Score is PAS=8.9+2.4×cg13230172-2.3×cg24434987

### Fruits & Vegetables Consumption Score (FVCS):

beta0 is comprised between about 0.7 and about 2
beta (1) is comprised between about 0.1 and about 1
beta (2) is comprised between about 0.5 and about 1.5

An example of a Fruits & vegetables consumption Score=1.1+0.4×cg12949927+1.0×cg15973528.

As a result, the Biological Age (BA) is determined or calculated as BA=beta (BA)+beta (BA1)×CpG(BA1)+beta (BA2)×CpG(BA2)+ . . . beta (BAi)×CpG(BAi),

An example of a Biological Age is BA=59.1+9.9×cg01844642-13.3×cg22156456+10.4×cg08097417+10.7×cg03545227+6.4×cg24724428-13.8×cg19722847-5.0×cg23753748-5.8×cg06885782-9.1×cg04474832+3.7×cg21899500+7.7×cg04084157

Preferably, any lifestyle related epigenetic signature as e.g. Tobacco smoking epigenetic signature can comprise predefined combinations of CpGs additionally to the randomly checked combinations or when a number of randomly chosen combinations of epigenetic signatures is defined, predefined CpGs can be added to said chosen combination.

The methylation status of at least three, preferably at least four, more preferably at least five, most preferably at least six of methylation sites of said epigenetic signature(s) is detected. In one aspect, the methylation status of all methylation sites is determined.

In an aspect of the invention, the methylation status of all four epigenetic signatures (i.e. tobacco and alcohol consumption, diet adequacy, and physical activity) is determined.

The methods described herein are computer implemented methods. The databases are stored in memory accessible from a processor in which a software is loaded to executed the method steps.

The invention further comprises a kit comprising probes for detecting the methylation status of at least two methylation sites selected from the list of Table 1 consisting of the Tobacco smoking epigenetic signature, the Alcohol drinking epigenetic signature, the Fruits & vegetables consumption epigenetic signature, and/or the Exercise epigenetic signature in a biological sample of said subject.

The present invention further provides a device comprising an analysis unit comprising means for implementing the methods for determining the youth capital of a subject.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein. Various references are cited throughout this Specification, each of which is incorporated herein by reference in its entirety. The foregoing description will be more fully understood with reference to the following Examples.

### Examples

Four Reference Populations were Used to Determine and Validate the Methods of the Invention.

The present examples calibrates epigenetic signatures with the SKIPOGH cohort, including 694 participants for whom genome wide methylation status, genome wide SNP, and lifestyle expositions were assessed.

The “GSE50660 dataset” was used to validate epigenetic signatures for age and for tobacco as well as a reference population provided by the applicant, comprising more than 100 persons, to estimate individual variability, repeatability and biological relevance. Furthermore GSI110043 as explained in connection with drawings was used.

A two-step approach was considered to determine the new metric of biological age. Firstly, we developed four specific signatures as indicators of external factors associated with lifestyle (exposure to tobacco, exposure to alcohol, fruits & vegetables consumption, and physical activity). Secondly, we determined the biological age as a combination of DNA methylation biomarkers conditional on such factors, epigenetic variability (DNA methylation biomarkers).

To select for meaningful biomarkers, we used the following approach
Identification of potential biomarkers from the literature (CpG methylation sites)
Identification of genetic confounding factors linked to methylation patterns (single nucleotide polymorphisms)
Random generation of millions of different markers combination (epigenetic signatures) associated with each exposition (multiple linear regression).

I Selection of combinations maximizing the biological relevance for each factor of interest (diet, physical activity, alcohol and tobacco consumption) with bayesian information criterion (BIC) and conditional goodness-of-fit.

To select for age biomarkers, we used a similar approach
Identification of potential biomarkers from the literature (CpG methylation sites)
Identification of genetic confounding factors linked to methylation patterns (single nucleotide polymorphisms)
Random generation of millions of different markers combination (epigenetic signatures) associated with chronological age (multiple linear regression).
Selection of combinations minimizing the difference between chronological age and estimated age, while maximizing the effects of lifestyle epigenetic signature diet, physical activity, alcohol and tobacco consumption) on the difference between chronological age and estimated age.
The resulting combinations are a trade-off between biological relevance and a good-fit between biological age and chronological age.

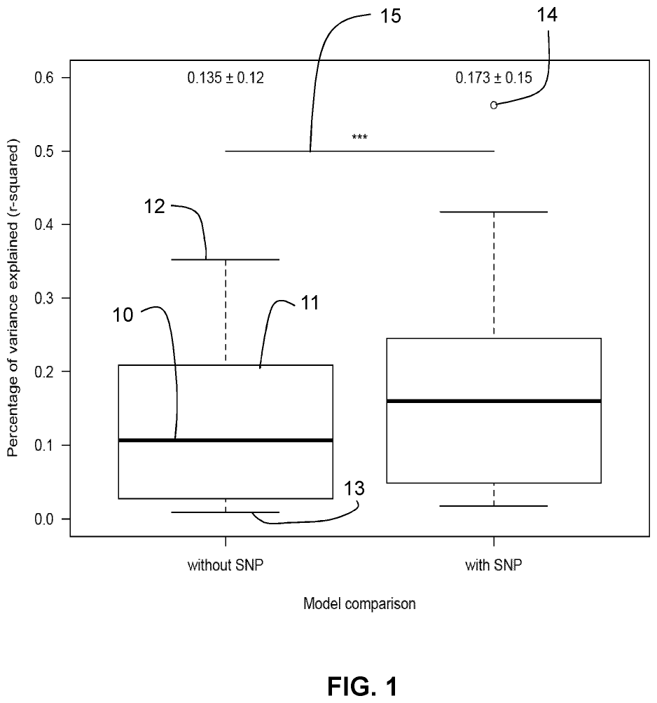

FIG. 1 shows a diagram of explained variance when the method according to an embodiment of the invention is applied with a SKIPOGH reference population. In other words, it shows the proportion of explained variance (R-squared) in successive exploratory linear regression models, among 689 participants of the Swiss adult population-based SKIPOGH study. The models included (left panel) either one CpG at a time (e.g., cg05575921, cg21566642, etc.; total number of tested CpGs=27. (right panel) comparing including one CpG alone and one CpG and its associated methyl-QTL SNP (number of tested SNPs=22). The diagrams are box-and-whiskers plots, with the darker line being the median 10 of the distribution, the box 11 represents the interquartile range, and the whiskers 12 and 13 represent the minimum and maximum value in the population (minus the outliers). Points that are outside the interquartile range times 2 are indeed considered outliers and specified as individual dots 14. The bar between the two diagrams represents a statistical test comparing the average 15 of the two distributions, the three stars indicate that the probability that the observed difference between the average of the two groups is due to random effects (p-value) is smaller than 0.001 (highly significant).

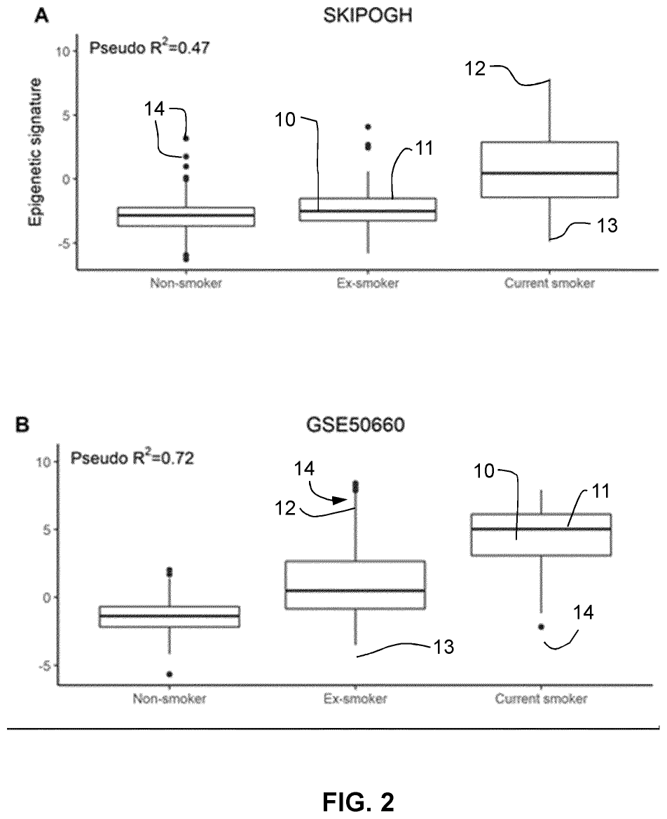

FIG. 2 shows two diagrams with a distribution of reference population categorized by smoking status for a method according to an embodiment of the invention. The distribution of the reference population categorized by smoking status in function of their epigenetic signature is shown in A using data from the SKIPOGH cohort as reference population, and is shown in B using data from the external validation cohort GSE50660. Reported pseudo-R2 values are from logistic regression models adjusting for age and sex. The diagrams are also box-and-whiskers plots, with the darker line being the median 10 of the distribution, the box 11 represents the interquartile range, and the whiskers represent the minimum 13 and maximum 12 value in the population (minus the outliers 14). Points that are outside the interquartile range times 2 are indeed considered outliers and specified as individual dots. The pseudo-R squared, represent the proportion of variance explained by the model.

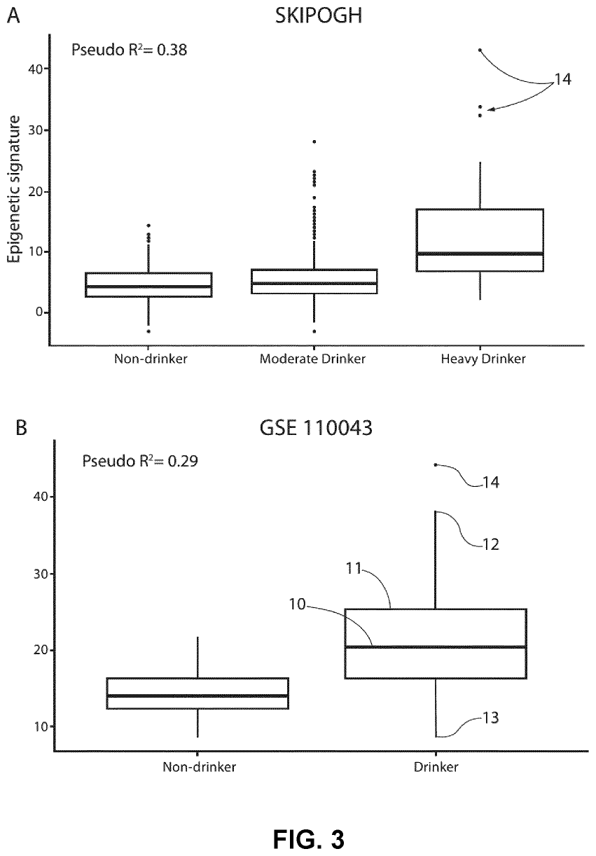

FIG. 3 shows two diagrams with a distribution of reference population categorized by drinking status for a method according to an embodiment of the invention. The distribution of the reference population participants are categorized by drinking status in function of their epigenetic signature wherein in A data from the SKIPOGH cohort as reference population is used, and wherein in B data from the external validation cohort GSE110043 is used. Reported pseudo-R2 values are from logistic regression models adjusting for age and sex (except for the GSE110043 cohort, which only includes sex). The diagrams are also box-and-whiskers plots, with the darker line 10 being the median of the distribution, the box 11 represents the interquartile range, and the whiskers represent the minimum 13 and maximum 12 value in the population (minus the outliers). Points that are outside the interquartile range times 2 are indeed considered outliers and specified as individual dots 14. The pseudo-R squared, represent the proportion of variance explained by the model.

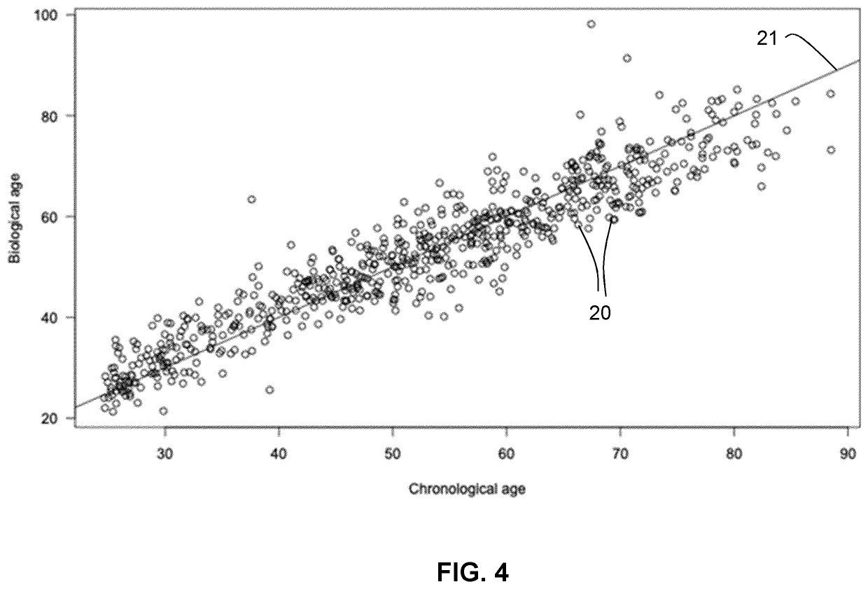

FIG. 4 shows a diagram between the chronological epigenetic age in the SKIPOGH reference population, i.e. the association between chronological age and epigenetic age in 694 participants of the Swiss adult population based SKIPOGH study. Each individual dot 20 represent one individual with his biological age plotted against his chronological age. The line 21 represents the equality between biological and chronological ages (Intercept=0, slope=1). The relationship between biological and chronological age has been assessed with a linear regression (p<0.001, R-squared=0.88).

### Reference Populations

### SKIPOGH Cohort

The epigenetic signatures were validated using data of 694 participants from the family-based multi-centric Swiss Kidney Project on Genes in Hypertension study (SKIPOGH) cohort, study procedures are described in details in¹⁰. Briefly, from 2009 to 2013, participants were recruited in three regions of Switzerland (in the cities of Lausanne, Geneva, and Bern) from a random population sample. Inclusion criteria were: aged≥18 years old; European ancestry; at least one first-degree family member willing to participate. Extensive information on tobacco smoking status was gathered from interview. Passive smoking was recorded as the average number of hours spent while exposed to cigarette smoke per day. Regarding alcohol intake, the average number of alcohol units per week was recorded (1 unit≈10 g of pure alcohol). The classification of drinking status (as heavy, moderate, or non-drinker) was calculated according to the Swiss federal public health guidelines regarding prevention of alcohol abuse 2018 (www.addictionsuisse.ch). The participation rate was 25.6%. Each region's local ethic committee approved the study protocol.

DNA from white blood cells was extracted using standard methods on a bead-based KingFisher Duo robot extraction system (ThermoFisher, Waltham, Massachusetts), and 1.2 ug of DNA were bisulfite-treated with EZ DNA Methylation@ Kit (Zymo Research). For the PCR step: alternative incubation conditions was performed when using the Illumina Infinium® Methylation Assay. The final elution was performed with 8 ul of M-Elution Buffer. DNA methylation levels were assessed by genome-wide DNA methylation micro-array platforms at respectively 450,000 and 850,000 loci by the Illumina HumanBeadChip 450K and EPIC 850K methylation arrays. Pre-processing was as follows: probes with detection p-values<10−16 were set to missing. Samples with a call rate<95% were excluded, and samples with swapped gender labels were removed if the swap could not be ascertained. Intensity values were corrected according to the background following the method provided by Illumina. Intensity values were quantile-normalized.

Six hundred ninety-four participants of the SKIPOGH study with non-missing data were included in the analysis. Principal characteristics of the participants are described in Table 3.

### GSE50660 Dataset

Tobacco and age were validated in another dataset of data from 464 Caucasian subjects participating in the CARDIOGENICS Consortium. This study recruited healthy individuals, along with patients suffering from coronary artery disease, aged between 38 and 67 (mean age=55.39 years, SD 6.6 years). Three centers (Paris, Cambridge, Leicester) were collecting participants' data from questionnaires and blood samples. DNA was extracted from whole blood using the DNeasy kit (Qiagen, Inc.). Bisulfite treatment of 750 ng of DNA was achieved with the 96 well EZ DNA Methylation kit (Zymo Research), in accordance with the manufacturer's instructions. The Infinium HumanMethylation450 K BeadChip (Illumina, Inc.) was used to assess DNA methylation levels at 485,577 cytosine positions in the human genome. Images intensities were analyzed using GenomeStudio software (2010.3), “methylation module” (1.8.5).

Identification of biomarkers: DNA methylation (CpG) and genetic variability (SNP): We first identified the potential biomarkers necessary to build epigenetic signatures. We considered as epigenetic biomarkers the most significant DNA methylation biomarkers previously identified from various sources, such as the EWAS atlas. Then, we selected the relevant methyl-QTLs identified in the literature associated with these methylation biomarkers. Combinations of methylation and methyl-QTL SNPs of the DNA methylation biomarkers were then included in the signatures.

528 methylation biomarkers associated with age or lifestyle factors (Table 2) were identified.

60 methyl-QTL SNPs associated with the methylation biomarkers were identified

### Reference Population GSE 110043 (Alcool)

This reference population was published on Apr. 1, 2018 as GSE 110043 and the title “Epigenome analysis of alcohol consumption in whole blood (WB) samples” for the Homo sapiens as organism. The samples were methylation profiled by genome tiling array and the genome wide DNA methylation profiling was performed for drinkers and non-drinkers in WB samples. The Illumina Infinium EPIC Human DNA methylation Beadchip was used to obtain DNA methylation profiles across 485,577 CpGs in WB samples that oevrlapped with CpGs from Illumina Infinium450k Human DNA methylation Beadchip. Samples included 47 drinkers (cases) and 47 non-drinkers (controls). Bisulfite converted DNA from the 94 samples were hybridized to the Illumina InfiniumEPIC Human Methylation Beadchip.

### SNP's Effects on Individual Variables

To validate the effectiveness of incorporating SNP's when estimating epigenetic signatures, we compared, within the SKIPOGH dataset, simple signatures using one CpG to signatures including one SNP/CpG combination. The Inventors identified 20 SNP/CpG combinations associated to age, 30 associated to tobacco consumption, and 2 associated to alcohol. They then compared the predictive power of the two approaches (CpG versus CpG+SNP) with chi-square-tests comparing residual sum of squares for each combination and also compared the overall distribution of both approaches with a paired-t-test.

They found that more than half of the combinations (27/52=52%) were better predictors of age, tobacco, or alcohol consumption (p-values lower than 0.05). Among these, the models including genetic variability (SNP) where on average 27% better at explaining the different factors (mean r-squared for CpG models: 0.135±0.12, mean r-squared for SNP/CpG models: 0.173+0.15, mean difference: 0.038, t26=4.31, p<0.001, FIG. 1).

These results indicate that the epigenetic response to environmental factors has a non-negligible genetic part and that epigenetic signatures accounting for genetic variability are better at describing the influence of external factors. Our epigenetic signatures are therefore more accurate than any previously published method.

### Epigenetic Signatures for External Factors

In order to design the epigenetic signatures for each participant, we built linear models including the most relevant CpGs identified for each trait along with the genotypes of all associated methyl-QTLs SNPs linked to these CpGs as covariates. Models were adjusted for age and sex whenever relevant. We performed linear models including the biomarkers as response variables and the trait as the outcome (e.g., current smoking status, current drinking status, weekly portions of fruit and vegetables).

We used conditional statistics to determine the best associations between CpGs and lifestyle traits. We used a three-step approach.
We built a goodness of fit distribution (R-squared) for potential SNP associations to each lifestyle trait (for example smoking status: current smoker or never smoker), by randomly associating CpGs to each lifestyle trait.
For each random association, we selected the most parsimonious model for each trait with Bayesian Information Criterion (BIC) stepwise regression in order to minimize the number of parameters.
For each parsimonious model, we used conditional modeling with alternative measures of exposition. We built alternative goodness-of-fit distribution that we used as a condition. For example with tobacco, we regressed the epigenetic scores derived from the smoking status to the number of cigarettes smoked, smoking duration, UPY (unit-pack year), and time since smoking cessation.

We defined the epigenetic signatures as the most parsimonious models that maximized the goodness-of-fit to the traits of interest and the conditional distributions.

All analyses were performed with R. Genomic references were made to the 19th version of the Human Genome assembly, accessed on the UCSC Genome Browser.

### Tobacco Smoking Epigenetic Signature

The effect of 241 CpG loci and 22 associated SNP (Table 2) on 423 participants that were either current smokers (N=174) or never smokers (N=249) was investigated.

We generated millions of individuals models on the 423 individuals with smoking status (current smoker or never smoker) as response variable and random combinations of CpG loci and associated SNPs as explanatory variables. Among these models, we selected the models that maximized, on all 694 participant within the SKIPOGH study, the association with: (1) daily cigarette consumption, (2) pack-year unit measuring the amount a person has smoked over a long period of time, (3) smoking time (in years since starting smoking) and (4) time since quitting (in years).

The 30 CpGs sites that were mostly associated with smoking variables are the following: cg05575921, cg26703534, cg08118908, cg01940273, cg14624207, cg15159987, cg23576855, cg14712058, cg21161138, cg07339236, cg00501876, cg21566642, cg23110422, cg05460226, cg01731783, cg03636183, cg17287155, cg21322436, cg25212025, cg04551776, cg09935388, cg19372602, cg03604011, cg14120703, cg01127300, cg13185177, cg04956244, cg00073090, cg01207684, cg12101586.

Some of the CpG sites included were associated with the ZNF385D gene (chr3, p24.3). Genetic polymorphisms of this gene are associated with numerous health-related outcomes, such as cardiovascular disease, bipolar disorder, cancer, etc. Two other CpGs are annotated to the AHRR gene, a gene coding for a protein that mediates dioxin toxicity and that interacts among other chemicals with benzo (a) pyrene, one of the carcinogens of tobacco smoke.

The best association of CpG and SNP, i.e. the epigenetic signature for tobacco consumption, was highly linked to the smoking status. Among SKIPOGH participants, the epigenetic signature for tobacco consumption was positively associated with self-reported tobacco consumption for current or never smokers (pseudo-R2=0.47). The explained variance was even higher for the GSE50660 validation cohort (pseudo-R2-0.72). When adjusting for age and sex, the epigenetic signature demonstrated a high capacity to distinguish between smokers and non-smokers in SKIPOGH (AUC=0.90; 95% CI=0.86-0.95).

When applying the epigenetic signatures to all of the 689 participants, we observed that current smokers are well and significantly differentiated from never smokers, as they have considerably higher signatures values than the latter (FIG. 2). We also see that individuals exposed to secondhand (passive) smoke and smokers that have quit, have higher signature values than never smokers. The signature variation within smokers is larger than the variation of never- and ex-smokers, representing the differences in smoking exposure among current smokers in terms of number of smoked cigarettes per day, intensities of smoking, length of smoking history, etc. Ex-smokers are located in-between current and never-smokers demonstrating first that the signature reflects the global (including past) smoking impact, and second that current smokers can expect to have an improvement of their test results when they quit smoking (FIG. 2).

### Alcohol Drinking Epigenetic Signature

We investigated the effect of 57 CpG loci and 2 associated SNP (Table 2) on 359 participants that were either Heavy drinkers (N=65) or non-drinkers (N=194). We generated hundreds of thousand individual models on the 359 individuals with drinking status (heavy drinker or non-drinker) as response variable and random combinations of CpG loci and associated SNPs as explanatory variables. Among these models, we selected the models that maximized, on all 694 participant within the SKIPOGH study, the association with: (1) weekly standard glass alcohol consumption, (2) drinking status (drinker, ex-drinker, or not drinking), and (3) drinking category (heavy, moderate, or non-drinker).

The 30 CpGs sites that were mostly associated with alcohol variables are the following: cg06690548, cg03497652, cg26248486, cg04987734, cg27241845, cg21566642, cg25998745, cg23975840, cg18336453, cg12873476, cg20970369, cg09448652, cg13127741, cg11376147, cg26213873, cg00716257, cg21626848, cg08677210, cg00622166, cg00271311, cg02711608, cg07502661, cg10317175, cg00291478, cg02003183, cg03329539, cg14476101, cg16246545, cg19238380, cg24859433 cg03497652 is annotated to the ANKS3 gene (chr16, p13.3). This gene interacts with several chemicals, including choline, and folic acid. cg06690548 is annotated to the SLC7A11 gene (chr4, q28.3), a gene associated with coronary heart disease and interacting with numerous chemicals, including alcohols. cg26248486 is on chr 12 (q21.2) and cg25998745 on chr 8 (q24.3), in the open sea DNA.

The best association of CpG and SNP (Table 2), i.e. the epigenetic signature for alcohol consumption, was highly linked to the drinking status (none, versus moderate, versus high drinker), SKIPOGH R2-0.24, GSE110043, pseudo-R2-0.29) (adjusting only for sex due to data availability).

When applying the epigenetic signatures to all the participants, we observe that, for both men and women, heavy alcohol drinkers are very well and statistically significantly differentiated from moderate drinkers, as they have considerably higher signatures values than the latter (FIG. 3). We also see that among individuals not currently drinking, those with a past drinking habits have higher signatures than those who were not drinking in the past. The variation of heavy and moderate drinkers is larger than the variation of non-drinkers, representing the differences in drinking exposure in terms of number of alcohol units, length of drinking history, etc.

### Fruits & Vegetables Consumption Epigenetic Signature:

We investigated the effect of 9 CpG loci on 687 participants that had estimated their weekly consumption of fruit and vegetable portions (between 0 and 8).

We generated all potential models on the 687 individuals with weekly portions of fruit and vegetables as response variable and random combinations of CpG loci as explanatory variables. Among these models, we selected the models that maximized, on all 694 participants within the SKIPOGH study, the association with: (1) BMI, (2) weight circumference, and (3) hip circumference.

The CpGs sites that were associated with diet variables are the following:
cg02211433, cg11643285, cg15973528, cg10335543, cg20926353, cg12949927, cg26047920, cg18156845, cg11955727 cg15973528 is annotated to the DYNC1H1 gene (chr14, q32.31), a gene associated with blood pressure and menopause, as well as interacting with numerous substances, including vitamins and micronutrients13. cg12949927 is annotated to the FHL2 gene (chr7, q11), a gene associated with smoking behavior and interacting with chemicals, such as benzo (a) pyrene.

The epigenetic signature for fruit and vegetable consumption, was mildly but highly significantly linked to the average number of fruits and vegetables units per day, and explained over 5% of the total variability (R-squared=0.057, F2, 684=20.6, p<0.001). The signature was also negatively associated with BMI (R-squared=0.024, F1, 689=17.0, p<0.001) and waist circumference (R-squared=0.089, F1, 688=67.2, p<0.001).

### Exercise Epigenetic Signature

We investigated the effect of 15 CpG loci on 663 participants for which we estimated the deviation from the expected percentage of body fat given age and sex, as a proxy to the amount of exercise.

We generated hundreds of thousand potential models on the 663 individuals with deviation from expected body fat as response variable and random combinations of CpG loci as explanatory variables. Among these models, we selected the models that maximized, on all 663 participants within the SKIPOGH study, the association with: (1) BMI, (2) weight circumference, and (3) hip circumference.

The CpGs sites that were associated with exercise variables are the following: cg02211433, cg11643285, cg15973528, cg10335543, cg20926353, cg12949927, cg26047920, cg18156845, cg11955727, cg01775802, cg13230172, cg11022537, cg20534702, cg02331198, cg24434987

The epigenetic signature for physical activity, was significantly linked to corrected body fat, and explained 2.0% of the total variability (R-squared=0.020, F2, 660=6.6, p<0.01). The signature was also negatively associated with BMI (R-squared=0.015, F1, 689=10.3, p<0.01) and waist circumference (R-squared=0.010, F1, 688=7.1, p<0.01).

### Epigenetic Signature for Biological Age

We investigated the effect of 190 CpG loci and 19 associated SNP on 694 participants.

We generated millions of individuals models with chronological age as response variable and random combinations of CpG loci and associated SNPs as explanatory variables. Among these models, we selected the models that maximized, on all participant within the SKIPOGH study, the association with: (1) each lifestyle signature derived from CpG information, (2) the combination of all four lifestyle signature, (3) the goodness-of-fit between chronological and biological age in the GSE50660 dataset.

The 60 CpGs sites that were mostly associated with age variables are the following: cg16867657, cg23606718, cg22454769, cg18450254, cg11693709, cg06493994, cg01820374, cg26161329, cg20822990, cg06639320, cg08415592, cg21120249, cg04875128, cg10501210, cg03365437, cg25427880, cg14556683, cg10189695, cg19283806, cg09118625, cg21709871, cg22736354, cg17110586, cg07211259, cg21899500, cg15195412, cg16477091, cg12079303, cg09809672, cg14692377, cg07082267, cg25478614, cg07080372, cg07408456, cg16419235, cg00565688, cg08370996, cg22947000, cg03607117, cg13836627, cg08957484, cg09559780, cg03399905, cg12934382, cg20264732, cg18902090, cg03972838, cg14956327, cg21186299, cg04453050, cg14918082, cg23078123, cg25410668, cg04084157, cg20692569, cg21296230, cg21801378, cg09547119, cg07553761, cg06782035 cg16867657 is annotated to the ELOVL2-AS1 gene (chr6, p24.2). cg22454769 and cg06639320 are both annotated to the FHL2 gene (chr2, q12.2), a gene associated with body weight and interacting with numerous chemicals, including alcohols. cg19283806 is annotated to the CCDCl02B gene (chr18, q22.1), a gene associated with body mass index, cholesterol levels, and interacting with zinc, aluminum, and arsenic. cg04875128 is annotated to the OTUD7A gene (chr15, q.13.3), a possible tumor suppressor gene, associated with mortality and interacting with multiple chemicals (acetaminophen, gentamicin, . . . ). cg02872426 and the SNP rs2003727 (intro variant, MAF (T)=0.35) are located in proximity on chr. 6 (q21) and annotated to the DDO gene, a gene associated with body weight, and interacting with multiple chemicals, including benzo (a) pyrene, and phenobarbital.

When accounting for conditional information, the remaining CpGs sites that were mostly associated with age are the following:
cg15195412, cg23753748, cg06885782, cg11299964, cg13836627, cg14209784, cg20822990, cg03020208, cg04036898, cg04084157, cg06782035, cg07211259, cg09809672, cg13899108, cg25268718, cg02046143, cg02650266, cg03032497, cg03224418, cg04474832, cg08622677, cg10189695, cg10523019, cg10804656, cg11084334, cg15480367, cg16386080, cg17497271, cg18573383, cg20426994

### Epigenetic Age and Chronological Age

The best association of CpG and SNP, i.e. the epigenetic signature for age, was significantly linked to chronological age, and explained 88.5% of the total variability (R-squared=0.885, F2, 692=5300, p<0.001). FIG. 4

### Epigenetic Age and Lifestyle Epigenetic Signature

We estimated the association between lifestyle epigenetic signature and biological age with a linear model with age signature as response variable and epigenetic signature as response variable. We then performed an analysis of variance on the model.

In the SKIPOGH study, we found a strong association between youth capital (difference between epigenetic age and chronological age) and lifestyle signatures, with epigenetic signature explaining 14% of the variability in youth capital (R-squared=0.14, F4, 689=28.7, p<0.001). We validated this in a second, open access dataset (GSE50660), in which we found the same association explaining 18% of the variability in youth capital (R-squared=0.18, F4, 459=24.6, p<0.001). In both dataset, all epigenetic signatures were strongly associated with biological age.

Youth Capital Comparison with Horvath and Hannum Biological Clocks

To compare our biological age estimation to the one already existing, we estimated the difference between biological age and chronological age (i.e. youth capital) and compared it between alternative metrics of biological age.

We found that our youth capital estimation (difference between biological and chronological age) is strongly linked to Horvath age acceleration metric (also the difference between biological and chronological age) in both the SKIPOGH dataset (R-squared=0.28, F_{1, 692}=270, p<0.001) and the GSE50660 dataset (R-squared=0.21, F_{1, 462}=122, p<0.001). This validates that our estimation of biological age is real as the other metrics have already been validated.

However, our estimation of youth capital is better to explain the importance of lifestyle than previous art, as it is explained at respectively 14% and 18% by lifestyle epigenetic signatures in the SKIPOGH and GSE50660 datasets, while Horvath age acceleration is only explained by 10% and 11% in both validation datasets, i.e. 30% to 60% less related to lifestyle than our scores. Same goes with the other metric of epigenetic age, Hannum's score, which is only related up to 8% and 3% to lifestyle epigenetic signatures.

Our youth capital is more linked to lifestyle than alternative scores existing. Our youth capital therefore better explains how lifestyle and it's change relate to epigenetic modifications and therefore has a better industrial potential.

### Signature Parsimony Compared to Other Metrics

Our estimation of biological age is based on a combination of 11 CpG sites, plus 16 for lifestyle signatures, i.e. a total of 27 loci. As a comparison, Horvath's biological clock has 353 age-associated CpGs, Hannum's has 71 age-associated CpGs, Levine's has 513 age-associated CpGs, and GrimAge has 1030 loci plus other metrics. Our parsimonious estimation allows for decreased costs and increased precision due to ease of multiplicate estimations.

TABLE 1

| | | Positions on | nucleotide |
|---|---|---|---|
| Epigenetic | Methylation | human | number of the |
| Signatures | sites | chromosome # | CpG sites |
| Tobacco smoking | cg05575921, | 5 | 373378 |
| epigenetic | cg26703534, | 5 | 377358 |
| signature | cg08118908, | 16 | 15787920 |
| | cg01940273, | 2 | 233284934 |
| | cg14624207, | 11 | 68142198 |
| | cg15159987, | 18 | 17003890 |
| | cg23576855, | 5 | 373299 |
| | cg14712058, | 19 | 16988083 |
| | cg21161138, | 5 | 399360 |
| | cg07339236, | 20 | 50312490 |
| | cg00501876, | 3 | 39193251 |
| | cg21566642, | 2 | 233284661 |
| | cg23110422, | 21 | 40182073 |
| | cg05460226, | 17 | 8804279 |
| | cg01731783, | 14 | 74211788 |
| | cg03636183, | 19 | 17000585 |
| | cg17287155, | 5 | 393347 |
| | cg21322436, | 7 | 145812842 |
| | cg25212025, | 10 | 34602937 |
| | cg04551776, | 5 | 393366 |
| | cg09935388, | 1 | 92947588 |
| | cg19372602, | 1 | 156116207 |
| | cg03604011, | 5 | 400201 |
| | cg14120703, | 9 | 139416102 |
| | cg01127300, | 22 | 38614796 |
| | cg13185177, | 3 | 194119885 |
| | cg04956244, | 17 | 38511592 |
| | cg00073090, | 19 | 1265879 |
| | cg01207684, | 16 | 4103167 |
| | cg12101586 | 15 | 75019203 |
| Fruits & | cg02211433, | 16 | 71496082 |
| vegetables | cg11643285, | 3 | 16411667 |
| consumption | cg15973528, | 14 | 102482817 |
| epigenetic | cg10335543, | 14 | 102829157 |
| signature | cg20926353, | 9 | 84303358 |
| | cg12949927, | 7 | 64298676 |
| | cg26047920, | 5 | 141303242 |
| | cg18156845, | 17 | 11985185 |
| | cg11955727 | 2 | 84105546 |
| Alcohol drinking | cg06690548, | 4 | 139162808 |
| epigenetic | cg03497652, | 16 | 4751569 |
| signature | cg26248486, | 12 | 76742361 |
| | cg04987734, | 14 | 103415873 |
| | cg27241845, | 2 | 233250370 |
| | cg21566642, | 2 | 233284661 |
| | cg25998745, | 8 | 142028625 |
| | cg23975840, | 12 | 117042895 |
| | cg18336453, | 6 | 43082296 |
| | cg12873476, | 8 | 142402728 |
| | cg20970369, | 1 | 111744108 |
| | cg09448652, | 11 | 62621367 |
| | cg13127741, | 20 | 31331821 |
| | cg11376147, | 11 | 57261198 |
| | cg26213873, | 1 | 112939056 |
| | cg00716257, | 14 | 75897417 |
| | cg21626848, | 17 | 39969267 |
| | cg08677210, | 17 | 55550613 |
| | cg00622166, | 16 | 46919021 |
| | cg00271311, | 11 | 58389290 |
| | cg02711608, | 19 | 47287964 |
| | cg07502661, | 2 | 43398339 |
| | cg10317175, | 10 | 25247855 |
| | cg00291478, | 10 | 121301041 |
| | cg02003183, | 14 | 103415882 |
| | cg03329539, | 2 | 233283329 |
| | cg14476101, | 1 | 120255992 |
| | cg16246545, | 1 | 120255941 |
| | cg19238380, | 1 | 156093948 |
| | cg24859433 | 6 | 30720203 |
| Exercise | cg02211433, | 16 | 71496082 |
| epigenetic | cg11643285, | 3 | 16411667 |
| signature | cg15973528, | 14 | 102482817 |
| | cg10335543, | 14 | 102829157 |
| | cg20926353, | 9 | 84303358 |
| | cg12949927, | 7 | 64298676 |
| | cg26047920, | 5 | 141303242 |
| | cg18156845, | 17 | 11985185 |
| | cg11955727, | 2 | 84105546 |
| | cg01775802, | 14 | 72945461 |
| | cg13230172, | 11 | 120195828 |
| | cg11022537, | 4 | 177738806 |
| | cg20534702, | 7 | 4752170 |
| | cg02331198, | 6 | 106988121 |
| | cg24434987 | 2 | 79221130 |
| Biological age | cg22736354 | 6 | 18122719 |
| | cg09809672 | 1 | 236557682 |
| | cg16867657 | 6 | 11044877 |
| | cg06493994 | 6 | 25652602 |
| | cg22454769 | 2 | 106015767 |
| | cg04084157 | 7 | 100809049 |
| | cg06639320 | 2 | 106015739 |
| | cg01820374 | 12 | 6882083 |
| | cg21296230 | 15 | 33010536 |
| | cg21801378 | 15 | 72612125 |
| | cg00059225 | 5 | 151304357 |
| | cg19761273 | 17 | 80232096 |
| | cg24724428 | 6 | 11044888 |
| | cg27320127 | 2 | 47798396 |
| | cg19722847 | 12 | 30849114 |
| | cg24079702 | 2 | 106015771 |
| | cg07553761 | 3 | 160167977 |
| | cg25427880 | 10 | 102322128 |
| | cg21572722 | 6 | 11044894 |
| | cg23606718 | 2 | 131513927 |
| | cg00481951 | 3 | 187387650 |
| | cg14692377 | 17 | 28562685 |
| | cg23124451 | 22 | 39548131 |
| | cg08090640 | 17 | 41159289 |
| | cg08097417 | 7 | 130419133 |
| | cg23500537 | 5 | 140419819 |
| | cg17861230 | 19 | 18343901 |
| | cg22947000 | 16 | 81272281 |
| | cg00503840 | 7 | 96650509 |
| | cg12373771 | 22 | 17601381 |
| | cg19560758 | 1 | 8086721 |
| | cg07547549 | 20 | 44658225 |
| | cg03032497 | 14 | 61108227 |
| | cg04528819 | 7 | 130418315 |
| | cg14674720 | 2 | 219827930 |
| | cg25410668 | 1 | 28241577 |
| | cg08415592 | 22 | 36648973 |
| | cg07164639 | 6 | 110736958 |
| | cg07388493 | 1 | 39491459 |
| | cg19996355 | 19 | 19729375 |
| | cg00563932 | 9 | 139871049 |
| | cg17110586 | 19 | 36454623 |
| | cg08209133 | 4 | 48485624 |
| | cg27553955 | 2 | 42720326 |
| | cg10501210 | 1 | 207997020 |
| | cg07082267 | 16 | 85429035 |
| | cg11071401 | 17 | 48637194 |
| | cg14361627 | 7 | 130419116 |
| | cg25809905 | 17 | 42467728 |
| | cg15195412 | 16 | 57406955 |
| | cg10137837 | 17 | 6926742 |
| | cg04474832 | 3 | 52008487 |
| | cg23091758 | 11 | 9025767 |
| | cg02228185 | 17 | 3379567 |
| | cg26614073 | 3 | 47517819 |
| | cg08234504 | 5 | 139013317 |
| | cg17471102 | 19 | 5851255 |
| | cg12261786 | 10 | 88727830 |
| | cg21899500 | 3 | 51740850 |
| | cg10917602 | 16 | 30996630 |
| | cg01844642 | 3 | 51989764 |
| | cg08160331 | 11 | 75140865 |
| | cg26161329 | 17 | 56832991 |
| | cg06291867 | 10 | 92617162 |
| | cg12946225 | 19 | 3573751 |
| | cg20692569 | 7 | 72848481 |
| | cg02650266 | 4 | 147558239 |
| | cg16419235 | 8 | 57360613 |
| | cg00439658 | 17 | 72848669 |
| | cg23753748 | 10 | 105212808 |
| | cg23995914 | 4 | 10459228 |
| | cg14956327 | 6 | 110737053 |
| | cg19724470 | 9 | 5450936 |
| | cg18473521 | 12 | 54448265 |
| | cg04427498 | 7 | 20830657 |
| | cg14556683 | 19 | 15342982 |
| | cg04836038 | 13 | 99739382 |
| | cg10523019 | 2 | 227700458 |
| | cg03224418 | 20 | 62611858 |
| | cg23517605 | 6 | 3228365 |
| | cg18236477 | 13 | 26043066 |
| | cg19283806 | 18 | 66389420 |
| | cg03607117 | 3 | 53080440 |
| | cg18573383 | 12 | 75603401 |
| | cg21120249 | 9 | 139921971 |
| | cg25148589 | 4 | 158141936 |
| | cg23320649 | 3 | 50604613 |
| | cg25090514 | 5 | 2038743 |
| | cg26394940 | 22 | 46449461 |
| | cg15297650 | 2 | 135477056 |
| | cg15201877 | 1 | 71512973 |
| | cg07408456 | 19 | 15590532 |
| | cg04875128 | 15 | 31775895 |
| | cg08622677 | 12 | 3601306 |
| | cg18902090 | 5 | 140306249 |
| | cg11220950 | 16 | 2042693 |
| | cg08262002 | 4 | 16575323 |
| | cg02046143 | 11 | 133797911 |
| | cg26290632 | 8 | 91094847 |
| | cg15341124 | 14 | 102027734 |
| | cg21709871 | 8 | 144923606 |
| | cg08957484 | 5 | 132083532 |
| | cg16717122 | 15 | 51973920 |
| | cg20669012 | 3 | 11102341 |
| | cg03020208 | 12 | 50354962 |
| | cg16146033 | 11 | 62767323 |
| | cg09118625 | 1 | 68512971 |
| | cg14314729 | 5 | 179815975 |
| | cg18267374 | 8 | 24771273 |
| | cg10189695 | 4 | 8582300 |
| | cg16386080 | 9 | 90589146 |
| | cg02613386 | 17 | 6679533 |
| | cg21870884 | 1 | 200842429 |
| | cg07211259 | 9 | 5510497 |
| | cg22919728 | 3 | 126242490 |
| | cg26337070 | 2 | 85999873 |
| | cg03365437 | 15 | 58357874 |
| | cg01763090 | 15 | 31775406 |
| | cg04453050 | 3 | 51740896 |
| | cg14918082 | 17 | 7833237 |
| | cg03545227 | 2 | 220173100 |
| | cg00462994 | 17 | 42081955 |
| | cg11084334 | 3 | 9594264 |
| | cg27210390 | 17 | 52978583 |
| | cg22273555 | 6 | 33130034 |
| | cg13682722 | 14 | 90798568 |
| | cg06458239 | 19 | 58038573 |
| | cg05923226 | 19 | 15121516 |
| | cg18450254 | 3 | 64200005 |
| | cg11693709 | 15 | 40542019 |
| | cg12079303 | 1 | 61547163 |
| | cg21186299 | 7 | 100808810 |
| | cg20822990 | 1 | 17338766 |
| | cg15957394 | 4 | 7941823 |
| | cg20052760 | 6 | 10510789 |
| | cg04845871 | 1 | 207996319 |
| | cg25316339 | 5 | 79866379 |
| | cg23341182 | 10 | 102046768 |
| | cg09462576 | 1 | 228297873 |
| | cg01441777 | 22 | 38714416 |
| | cg09547119 | 19 | 52391367 |
| | cg00565688 | 1 | 3568212 |
| | cg06885782 | 1 | 41248209 |
| | cg15538427 | 11 | 62457014 |
| | cg13899108 | 19 | 18344322 |
| | cg17497271 | 15 | 40212781 |
| | cg01797043 | 16 | 2004686 |
| | cg26921969 | 5 | 92948217 |
| | cg19784428 | 19 | 16830746 |
| | cg18064714 | 7 | 20824556 |
| | cg11705975 | 10 | 120354248 |
| | cg25256723 | 1 | 169555944 |
| | cg12534424 | 7 | 127992316 |
| | cg07850604 | 14 | 36003443 |
| | cg16969368 | 17 | 57642752 |
| | cg14676592 | 16 | 49910862 |
| | cg03399905 | 15 | 79576060 |
| | cg24436906 | 2 | 242498081 |
| | cg12554573 | 3 | 51976667 |
| | cg04036898 | 1 | 46664598 |
| | cg06874016 | 17 | 40177415 |
| | cg14209784 | 10 | 88729861 |
| | cg10804656 | 10 | 22623460 |
| | cg22016779 | 2 | 230452311 |
| | cg02872426 | 6 | 110736772 |
| | cg23078123 | 1 | 68577796 |
| | cg18240400 | 10 | 46168597 |
| | cg00484358 | 1 | 110610995 |
| | cg22809047 | 2 | 101618261 |
| | cg15845821 | 19 | 16830613 |
| | cg03972838 | 22 | 39897580 |
| | cg20119148 | 19 | 18344195 |
| | cg25478614 | 3 | 187387866 |
| | cg01511567 | 11 | 57103631 |
| | cg20264732 | 16 | 68269763 |
| | cg09559780 | 16 | 50727221 |
| | cg25268718 | 14 | 24604711 |
| | cg12934382 | 3 | 51741135 |
| | cg20249566 | 19 | 16830739 |
| | cg15480367 | 14 | 93389485 |
| | cg05991454 | 4 | 147558435 |
| | cg07080372 | 11 | 796607 |
| | cg26685941 | 13 | 95952902 |
| | cg06335143 | 1 | 53308654 |
| | cg11299964 | 9 | 128469783 |
| | cg13836627 | 15 | 30113723 |
| | cg16290275 | 1 | 208042910 |
| | cg20426994 | 7 | 130418324 |
| | cg16541931 | 10 | 25463757 |
| | cg01560871 | 10 | 72545424 |
| | cg19885761 | 5 | 175223646 |
| | cg08370996 | 15 | 96874031 |
| | cg17421623 | 3 | 119187570 |
| | cg16015712 | 1 | 53308597 |
| | cg00664406 | 3 | 51740875 |
| | cg16477091 | 17 | 56833000 |
| | cg07502389 | 8 | 24771259 |
| | cg05156137 | 21 | 35898975 |
| | cg16867657 | 6 | 11044877 |
| | cg06782035 | 5 | 16179135 |

TABLE 2

| | | | Nucleotide | | | | | | |
|---|---|---|---|---|---|---|---|---|---|
| | | Human | number of | | | | CpG Value for allele | | |
| CpG | methyl-QTL | chromo- | the SNP | SNP combination | | | combination | | |
| associated | SNP | some # | position | #1 | #2 | #3 | #1 | #2 | #3 |
| cg00336149 | rs260500 | 19 | 58791213 | AA | AC | CC | 0.4749 | 0.4369 | 0.4165 |
| cg01511567 | rs11228995 | 11 | 57083535 | CC | CT | TT | 0.1659 | 0.1984 | 0.2362 |
| cg02872426 | rs3757351 | 6 | 110735630 | AA | AG | GG | 0.6181 | 0.5171 | 0.4167 |
| cg03224418 | rs817336 | 20 | 62603060 | CC | CG | GG | 0.4331 | 0.4198 | 0.3981 |
| cg03531211 | rs13332669 | 16 | 10998995 | AA | AT | TT | 0.6004 | 0.6128 | 0.6110 |
| cg03604011 | rs76293297 | 8 | 18526242 | AA | AC | NA | 0.0709 | 0.0611 | NA |
| cg03972838 | rs2009774 | 22 | 39879679 | AA | AT | TT | 0.6178 | 0.6244 | 0.6409 |
| cg05156137 | rs2284576 | 21 | 35914812 | AA | AG | GG | 0.3263 | 0.3339 | 0.3539 |
| cg05329352 | rs12161672 | 10 | 112887471 | CC | CG | GG | 0.6392 | 0.5952 | 0.5659 |
| cg05923226 | rs10854136 | 19 | 15029174 | AA | AG | GG | 0.6640 | 0.6298 | 0.6027 |
| cg06121808 | rs908545 | 2 | 113389232 | AA | AG | GG | 0.4667 | 0.4182 | 0.3922 |
| cg06885782 | rs2769255 | 1 | 41245354 | CC | CT | TT | 0.5953 | 0.6113 | 0.6215 |
| cg07164639 | rs2003727 | 6 | 110738291 | CC | CT | TT | 0.3735 | 0.4473 | 0.5152 |
| cg09118625 | rs12073947 | 1 | 68363490 | AA | AC | CC | 0.6284 | 0.6395 | 0.6667 |
| cg09658497 | rs7790322 | 7 | 2830498 | CC | CT | TT | 0.7198 | 0.6457 | 0.5734 |
| cg10917602 | rs12934900 | 16 | 30923602 | AA | AT | TT | 0.6875 | 0.7014 | 0.7214 |
| cg11207515 | rs7783508 | 7 | 146897589 | CC | CT | TT | 0.4045 | 0.4332 | 0.4506 |
| cg12803068 | rs61087358 | 7 | 45017743 | AA | AG | GG | 0.6716 | 0.7344 | 0.7786 |
| cg14314729 | rs4700950 | 5 | 179825931 | AA | AG | GG | 0.4648 | 0.4606 | 0.4444 |
| cg14476101 | rs11583993 | 1 | 120255370 | AA | AG | GG | 0.5132 | 0.5968 | 0.6583 |
| cg14918082 | rs8070212 | 17 | 7829928 | AA | AG | GG | 0.8007 | 0.7821 | 0.7625 |
| cg14956327 | rs3757351 | 6 | 110735630 | AA | AG | GG | 0.3974 | 0.3478 | 0.2959 |
| cg15417641 | rs260486 | 19 | 58753937 | CC | CT | TT | 0.6909 | 0.6546 | 0.6155 |
| cg15542713 | rs2147904 | 1 | 42371414 | CC | CT | TT | 0.5759 | 0.5407 | 0.5054 |
| cg15693572 | rs9845701 | 3 | 22368388 | CC | CG | GG | 0.5217 | 0.5677 | 0.6094 |
| cg16145216 | rs2147904 | 1 | 42371414 | CC | CT | TT | 0.3968 | 0.3740 | 0.3435 |
| cg16246545 | rs11583993 | 1 | 120255370 | AA | AG | GG | 0.3837 | 0.4615 | 0.5120 |
| cg17272563 | rs186587921 | 1 | 43925902 | AC | CC | NA | 0.2039 | 0.2029 | NA |
| cg17421623 | rs62263406 | 3 | 119165162 | AA | AG | GG | 0.4530 | 0.3850 | 0.3127 |
| cg18240400 | rs76831684 | 10 | 46116032 | CC | CT | TT | 0.4968 | 0.5488 | 0.6108 |
| cg18316974 | rs115401860 | 1 | 92362377 | AA | AG | NA | 0.9328 | 0.8295 | NA |
| cg18369990 | rs4073745 | 5 | 176733341 | AA | AG | GG | 0.5838 | 0.5839 | 0.5911 |
| cg19093370 | rs146263864 | 16 | 52005011 | AG | GG | NA | 0.7788 | 0.7784 | NA |
| cg19382157 | rs4721325 | 7 | 2069846 | AA | AG | GG | 0.5160 | 0.4557 | 0.2969 |
| cg19717773 | rs7790322 | 7 | 2830498 | CC | CT | TT | 0.6719 | 0.6141 | 0.5564 |
| cg19724470 | rs10975133 | 9 | 5486390 | GG | GT | TT | 0.2080 | 0.2565 | 0.2970 |
| cg20066188 | rs727047 | 22 | 37677719 | AA | AG | GG | 0.4256 | 0.3855 | 0.3464 |
| cg20303561 | rs28651318 | 14 | 91999791 | AA | AG | GG | 0.7136 | 0.6938 | 0.6655 |
| cg21188533 | rs260452 | 19 | 58766874 | AA | AC | CC | 0.5601 | 0.6101 | 0.6573 |
| cg22132788 | rs61087358 | 7 | 45017743 | AA | AG | GG | 0.8218 | 0.8443 | 0.8867 |
| cg23480021 | rs7631738 | 3 | 22610981 | CC | CT | TT | 0.6705 | 0.6577 | 0.6829 |
| cg23681440 | rs7997900 | 13 | 27293625 | CC | CT | TT | 0.4651 | 0.4565 | 0.4396 |
| cg24049493 | rs2147904 | 1 | 42371414 | CC | CT | TT | 0.3122 | 0.2721 | 0.2360 |
| cg24172324 | rs13025087 | 2 | 232264914 | GG | GT | TT | 0.4266 | 0.3887 | 0.3583 |
| cg25212025 | rs1985313 | 10 | 34580880 | GG | GT | TT | 0.4476 | 0.4186 | 0.3876 |
| cg25809905 | rs118180322 | 17 | 42645948 | CC | CT | NA | 0.6286 | 0.6888 | NA |
| cg26337070 | rs12478164 | 2 | 85992977 | AA | AT | TT | 0.6680 | 0.6372 | 0.6159 |
| cg26394940 | rs12484609 | 22 | 46450297 | CC | CT | TT | 0.1082 | 0.1422 | 0.1847 |
| cg26614073 | rs115360749 | 3 | 46713120 | AG | GG | NA | 0.5121 | 0.5212 | NA |
| cg26718213 | rs12464946 | 2 | 242002701 | CC | CG | GG | 0.3834 | 0.3391 | 0.3158 |

TABLE 3
Principal characteristics of the 694 SKIPOGH participants

| | Women | | Men | |
|---|---|---|---|---|
| Variables | (n = 359) | | (n = 335) | |
| Center Lausanne | 169 | | 141 | |
| Center Geneva | 140 | | 141 | |
| Center Bern | 50 | | 53 | |
| Age [years] (mean, SD, range) | 52.9 (15.1, | | 52.5 (15.9, | |
| | 24.9-85.4) | | 24.6-88.5) | |
| Body mass index [kg/m\hat{ }2] (mean, SD) | 24.7 | (4.9) | 26.6 | (4.2) |
| Smoking status: (3 missing answers in | | | | |
| women, 2 in men) | | | | |
| Currently tobacco smoker (n, %) | 76 | (21.3) | 98 | (29.7) |
| Smoked cigarettes/day [in current | 7 | (9.6) | 7.5 | (9) |
| smokers] (mean, SD) | | | | |
| Never tobacco smoker (n, %) | 146 | (41) | 103 | (31.2) |
| Ex-tobacco smoker (n, %) | 99 | (27.8) | 102 | (30.9) |
| Exposed to passive tobacco smoke at | 35 | (9.8) | 27 | (8.2) |
| least 1 h/d (n, %) | | | | |
| Alcohol status: (0 missing answers) | | | | |
| Heavy alcohol drinkers (n, %) | 28 | (7.8) | 37 | (11) |
| Alcohol unit(s) drinked per week by | 19.8 | (4.1) | 33.4 | (11.6) |
| heavy drinkers (mean, SD) | | | | |
| Moderate alcohol drinkers (n, %) | 180 | (50.1) | 233 | (69.6) |
| Alcohol unit(s) drinked per week by | 4.6 | (3.6) | 7.5 | (5.2) |
| moderate drinkers (mean, SD) | | | | |
| Not currently drinking alcohol (n, %) | 151 | (42.1) | 65 | (19.4) |
| Of which are ex-drinkers (n, %) | 12 | (7.9) | 10 | (15.4) |
| Number of portions of fruits/day (mean, | 2 | (0.8) | 1.7 | (0.8) |
| SD) (1 missing answers for women, 4 | | | | |
| for men) | | | | |
| Number of portions of vegetables/day | 2.2 | (0.7) | 1.9 | (0.7) |
| (mean, SD) (1 missing answers for | | | | |
| women, 4 for men) | | | | |
| Number of portions of either fruits or | 4.2 | (1.2) | 3.7 | (1.3) |
| vegetables (mean, SD) (2 missing | | | | |
| answers for women, 5 for men) | | | | |

### LIST OF REFERENCE SIGNS

10 median of distribution
11 interquartile range
12 maximum value
13 minimum value
14 outlier dot
15 average of distributions
20 individual's biological against chronological age
21 equality between biological and chronological age

1-9. (canceled)

10. A computer implemented method determining the difference between the biological age and the chronological age of a subject U in connection with the influence of measurable lifestyle factors, wherein the method comprises the steps of:
a.) providing access to a database of a reference population having n_{BA} members, wherein the database comprises:
n^{ba}ₘₛ methylation levels (CpG(i,1), . . . ,CpG(i,n^{ba}ₘₛ) for i=1 to n_{BA} of a plurality of predetermined methylation sites (CpG(1) . . . . CpG(n^{ba}ₘₛ)) of human cells related to the biological age for the n_{BA} members of said reference population,
n^{ls}ₘₛ methylation levels (CpG(i,1) . . . . CpG(i,n^{ls}ₘₛ)) for i=1 to n_{BA} of a plurality of methylation sites (CpG(1) . . . . CpG(n^{ls}ₘₛ)) of human cells related to life style factors for the n_{BA} members of said reference population, and
the chronological age of each member of the reference population,
b.) choosing a number p for the number of subsets Sₘₛ to be evaluated,
c.) choosing a subset Sₘₛ of combinations of mₚ methylation sites selected from the database with for each set comprising one or more different CpG(j) ∈ Sₘₛ with j taking predetermined values j ∈ {1, 2, . . . , mₚ} of the chosen combination,
d.) calculating the biological age BA (i) for each member i of said reference population, i=1 to n_{BA}, for the set 1 out of the p Sₘₛ of combinations with the formula:
$$BA ( i ) = {beta}_{0} + \sum {beta}_{j} \times CpG ( i , j ) ,$$
with CpG(i,j) being the methylation level for the member i and methylation site j and betaj being a parameter multiplying the methylation level of the associated CpG(j),
while
the prediction of chronological age for the members i of the reference population is maximized with general linear models,
followed by maximizing the proportion of variability in the difference between biological age and chronological age with conditional (Bayesian) statistics for the members of reference population for the subset Si for each of the—one to four-chosen epigenetic signatures ES's of the life style factors as well as preferably a combination of thereof,
e.) checking if the value of the subset i has reached the maximum number p of subsets to be evaluated and if not, raising the subset number by one and going back to method step c.) and if yes, continuing with next step f.),
f.) calculating a combined selection value based on the maximized chronological age and the variability value of the life style factor values represented by the chosen epigenetic signatures ES^{ls} for each life style value and preferably a combination of the four life style values with a polynominal function,
g.) determining the set Sₘₐₓ out of the p sets Sₘₛ with the maximized combined selection value having the parameters beta₀ and betaj with 1 taking predetermined values 1 € {1, 2, . . . , mₚ} of the determined set Sₘₐₓ,
h.) providing methylation levels (CpG(S,1) . . . . CpG(S,n^{ls}ₘₛ)) of human cells related to a subject U for all methylation sites related to biological age factors chosen in the set Sₘₐₓ,
i.) determining the biological age of said subject U as
$$BA ( S ) = {beta}_{0} + \sum {beta}_{l} \times CpG ( S , l ) ,$$
with CpG(S,1) being the methylation level for the subject U and methylation site 1 from the determined set Sₘₐₓ, and
j.) determining the difference between the biological age calculated in i.) and the chronological age of said subject U wherein said difference results is the youth capital.

11. The method according to claim 10, wherein each set out of the p sets Sₘₛ comprises a combination between 10 to 50 methylation sites.

12. The method according to claim 11, wherein the number p of sets used in method step c.) and d.) as combinations from the n^{ba}ₘₛ possible methylation sites is chosen as p equal to the binomial coefficient “n^{ba}ₘₛ choose k”, wherein n^{ba}ₘₛ>=k>=10.

13. The method according to claim 10, wherein the methylation sites of human cells related to life style factors comprise one or more methylation sites of the Tobacco smoking epigenetic signature, of the Alcohol drinking epigenetic signature, of the Fruits & vegetables consumption epigenetic signature, and/or of the Exercise epigenetic signature in said biological sample of a reference population.

14. The method according to claim 13, wherein the methylation sites related to the life style factors are selected from the list of life style factor methylation sites.

| | | Positions on | |
|---|---|---|---|
| Epigenetic | Methylation | human | nucleotide number |
| Signatures | sites | chromosome # | of the CpG sites |
| Tobacco smoking | cg05575921, | 5 | 373378 |
| epigenetic | cg26703534, | 5 | 377358 |
| signature | cg08118908, | 16 | 15787920 |
| | cg01940273, | 2 | 233284934 |
| | cg14624207, | 11 | 68142198 |
| | cg15159987, | 18 | 17003890 |
| | cg23576855, | 5 | 373299 |
| | cg14712058, | 19 | 16988083 |
| | cg21161138, | 5 | 399360 |
| | cg07339236, | 20 | 50312490 |
| | cg00501876, | 3 | 39193251 |
| | cg21566642, | 2 | 233284661 |
| | cg23110422, | 21 | 40182073 |
| | cg05460226, | 17 | 8804279 |
| | cg01731783, | 14 | 74211788 |
| | cg03636183, | 19 | 17000585 |
| | cg17287155, | 5 | 393347 |
| | cg21322436, | 7 | 145812842 |
| | cg25212025, | 10 | 34602937 |
| | cg04551776, | 5 | 393366 |
| | cg09935388, | 1 | 92947588 |
| | cg19372602, | 1 | 156116207 |
| | cg03604011, | 5 | 400201 |
| | cg14120703, | 9 | 139416102 |
| | cg01127300, | 22 | 38614796 |
| | cg13185177, | 3 | 194119885 |
| | cg04956244, | 17 | 38511592 |
| | cg00073090, | 19 | 1265879 |
| | cg01207684, | 16 | 4103167 |
| | cg12101586 | 15 | 75019203 |
| Fruits & | cg02211433, | 16 | 71496082 |
| vegetables | cg11643285, | 3 | 16411667 |
| consumption | cg15973528, | 14 | 102482817 |
| epigenetic | cg10335543, | 14 | 102829157 |
| signature | cg20926353, | 9 | 84303358 |
| | cg12949927, | 7 | 64298676 |
| | cg26047920, | 5 | 141303242 |
| | cg18156845, | 17 | 11985185 |
| | cg11955727 | 2 | 84105546 |
| Alcohol drinking | cg06690548, | 4 | 139162808 |
| epigenetic | cg03497652, | 16 | 4751569 |
| signature | cg26248486, | 12 | 76742361 |
| | cg04987734, | 14 | 103415873 |
| | cg27241845, | 2 | 233250370 |
| | cg21566642, | 2 | 233284661 |
| | cg25998745, | 8 | 142028625 |
| | cg23975840, | 12 | 117042895 |
| | cg18336453, | 6 | 43082296 |
| | cg12873476, | 8 | 142402728 |
| | cg20970369, | 1 | 111744108 |
| | cg09448652, | 11 | 62621367 |
| | cg13127741, | 20 | 31331821 |
| | cg11376147, | 11 | 57261198 |
| | cg26213873, | 1 | 112939056 |
| | cg00716257, | 14 | 75897417 |
| | cg21626848, | 17 | 39969267 |
| | cg08677210, | 17 | 55550613 |
| | cg00622166, | 16 | 46919021 |
| | cg00271311, | 11 | 58389290 |
| | cg02711608, | 19 | 47287964 |
| | cg07502661, | 2 | 43398339 |
| | cg10317175, | 10 | 25247855 |
| | cg00291478, | 10 | 121301041 |
| | cg02003183, | 14 | 103415882 |
| | cg03329539, | 2 | 233283329 |
| | cg14476101, | 1 | 120255992 |
| | cg16246545, | 1 | 120255941 |
| | cg19238380, | 1 | 156093948 |
| | cg24859433 | 6 | 30720203 |
| Exercise | cg02211433, | 16 | 71496082 |
| epigenetic | cg11643285, | 3 | 16411667 |
| signature | cg15973528, | 14 | 102482817 |
| | cg10335543, | 14 | 102829157 |
| | cg20926353, | 9 | 84303358 |
| | cg12949927, | 7 | 64298676 |
| | cg26047920, | 5 | 141303242 |
| | cg18156845, | 17 | 11985185 |
| | cg11955727, | 2 | 84105546 |
| | cg01775802, | 14 | 72945461 |
| | cg13230172, | 11 | 120195828 |
| | cg11022537, | 4 | 177738806 |
| | cg20534702, | 7 | 4752170 |
| | cg02331198, | 6 | 106988121 |
| | cg24434987 | 2 | 79221130 |

15. The method according to claim 13, wherein the methylation sites related to the biological age are selected from the list of marked biological age methylation sites.

| | | Positions on | |
|---|---|---|---|
| Epigenetic | Methylation | human | nucleotide number |
| Signatures | sites | chromosome # | of the CpG sites |
| Biological age | cg22736354 | 6 | 18122719 |
| | cg09809672 | 1 | 236557682 |
| | cg16867657 | 6 | 11044877 |
| | cg06493994 | 6 | 25652602 |
| | cg22454769 | 2 | 106015767 |
| | cg04084157 | 7 | 100809049 |
| | cg06639320 | 2 | 106015739 |
| | cg01820374 | 12 | 6882083 |
| | cg21296230 | 15 | 33010536 |
| | cg21801378 | 15 | 72612125 |
| | cg00059225 | 5 | 151304357 |
| | cg19761273 | 17 | 80232096 |
| | cg24724428 | 6 | 11044888 |
| | cg27320127 | 2 | 47798396 |
| | cg19722847 | 12 | 30849114 |
| | cg24079702 | 2 | 106015771 |
| | cg07553761 | 3 | 160167977 |
| | cg25427880 | 10 | 102322128 |
| | cg21572722 | 6 | 11044894 |
| | cg23606718 | 2 | 131513927 |
| | cg00481951 | 3 | 187387650 |
| | cg14692377 | 17 | 28562685 |
| | cg23124451 | 22 | 39548131 |
| | cg08090640 | 17 | 41159289 |
| | cg08097417 | 7 | 130419133 |
| | cg23500537 | 5 | 140419819 |
| | cg17861230 | 19 | 18343901 |
| | cg22947000 | 16 | 81272281 |
| | cg00503840 | 7 | 96650509 |
| | cg12373771 | 22 | 17601381 |
| | cg19560758 | 1 | 8086721 |
| | cg07547549 | 20 | 44658225 |
| | cg03032497 | 14 | 61108227 |
| | cg04528819 | 7 | 130418315 |
| | cg14674720 | 2 | 219827930 |
| | cg25410668 | 1 | 28241577 |
| | cg08415592 | 22 | 36648973 |
| | cg07164639 | 6 | 110736958 |
| | cg07388493 | 1 | 39491459 |
| | cg19996355 | 19 | 19729375 |
| | cg00563932 | 9 | 139871049 |
| | cg17110586 | 19 | 36454623 |
| | cg08209133 | 4 | 48485624 |
| | cg27553955 | 2 | 42720326 |
| | cg10501210 | 1 | 207997020 |
| | cg07082267 | 16 | 85429035 |
| | cg11071401 | 17 | 48637194 |
| | cg14361627 | 7 | 130419116 |
| | cg25809905 | 17 | 42467728 |
| | cg15195412 | 16 | 57406955 |
| | cg10137837 | 17 | 6926742 |
| | cg04474832 | 3 | 52008487 |
| | cg23091758 | 11 | 9025767 |
| | cg02228185 | 17 | 3379567 |
| | cg26614073 | 3 | 47517819 |
| | cg08234504 | 5 | 139013317 |
| | cg17471102 | 19 | 5851255 |
| | cg12261786 | 10 | 88727830 |
| | cg21899500 | 3 | 51740850 |
| | cg10917602 | 16 | 30996630 |
| | cg01844642 | 3 | 51989764 |
| | cg08160331 | 11 | 75140865 |
| | cg26161329 | 17 | 56832991 |
| | cg06291867 | 10 | 92617162 |
| | cg12946225 | 19 | 3573751 |
| | cg20692569 | 7 | 72848481 |
| | cg02650266 | 4 | 147558239 |
| | cg16419235 | 8 | 57360613 |
| | cg00439658 | 17 | 72848669 |
| | cg23753748 | 10 | 105212808 |
| | cg23995914 | 4 | 10459228 |
| | cg14956327 | 6 | 110737053 |
| | cg19724470 | 9 | 5450936 |
| | cg18473521 | 12 | 54448265 |
| | cg04427498 | 7 | 20830657 |
| | cg14556683 | 19 | 15342982 |
| | cg04836038 | 13 | 99739382 |
| | cg10523019 | 2 | 227700458 |
| | cg03224418 | 20 | 62611858 |
| | cg23517605 | 6 | 3228365 |
| | cg18236477 | 13 | 26043066 |
| | cg19283806 | 18 | 66389420 |
| | cg03607117 | 3 | 53080440 |
| | cg18573383 | 12 | 75603401 |
| | cg21120249 | 9 | 139921971 |
| | cg25148589 | 4 | 158141936 |
| | cg23320649 | 3 | 50604613 |
| | cg25090514 | 5 | 2038743 |
| | cg26394940 | 22 | 46449461 |
| | cg15297650 | 2 | 135477056 |
| | cg15201877 | 1 | 71512973 |
| | cg07408456 | 19 | 15590532 |
| | cg04875128 | 15 | 31775895 |
| | cg08622677 | 12 | 3601306 |
| | cg18902090 | 5 | 140306249 |
| | cg11220950 | 16 | 2042693 |
| | cg08262002 | 4 | 16575323 |
| | cg02046143 | 11 | 133797911 |
| | cg26290632 | 8 | 91094847 |
| | cg15341124 | 14 | 102027734 |
| | cg21709871 | 8 | 144923606 |
| | cg08957484 | 5 | 132083532 |
| | cg16717122 | 15 | 51973920 |
| | cg20669012 | 3 | 11102341 |
| | cg03020208 | 12 | 50354962 |
| | cg16146033 | 11 | 62767323 |
| | cg09118625 | 1 | 68512971 |
| | cg14314729 | 5 | 179815975 |
| | cg18267374 | 8 | 24771273 |
| | cg10189695 | 4 | 8582300 |
| | cg16386080 | 9 | 90589146 |
| | cg02613386 | 17 | 6679533 |
| | cg21870884 | 1 | 200842429 |
| | cg07211259 | 9 | 5510497 |
| | cg22919728 | 3 | 126242490 |
| | cg26337070 | 2 | 85999873 |
| | cg03365437 | 15 | 58357874 |
| | cg01763090 | 15 | 31775406 |
| | cg04453050 | 3 | 51740896 |
| | cg14918082 | 17 | 7833237 |
| | cg03545227 | 2 | 220173100 |
| | cg00462994 | 17 | 42081955 |
| | cg11084334 | 3 | 9594264 |
| | cg27210390 | 17 | 52978583 |
| | cg22273555 | 6 | 33130034 |
| | cg13682722 | 14 | 90798568 |
| | cg06458239 | 19 | 58038573 |
| | cg05923226 | 19 | 15121516 |
| | cg18450254 | 3 | 64200005 |
| | cg11693709 | 15 | 40542019 |
| | cg12079303 | 1 | 61547163 |
| | cg21186299 | 7 | 100808810 |
| | cg20822990 | 1 | 17338766 |
| | cg15957394 | 4 | 7941823 |
| | cg20052760 | 6 | 10510789 |
| | cg04845871 | 1 | 207996319 |
| | cg25316339 | 5 | 79866379 |
| | cg23341182 | 10 | 102046768 |
| | cg09462576 | 1 | 228297873 |
| | cg01441777 | 22 | 38714416 |
| | cg09547119 | 19 | 52391367 |
| | cg00565688 | 1 | 3568212 |
| | cg06885782 | 1 | 41248209 |
| | cg15538427 | 11 | 62457014 |
| | cg13899108 | 19 | 18344322 |
| | cg17497271 | 15 | 40212781 |
| | cg01797043 | 16 | 2004686 |
| | cg26921969 | 5 | 92948217 |
| | cg19784428 | 19 | 16830746 |
| | cg18064714 | 7 | 20824556 |
| | cg11705975 | 10 | 120354248 |
| | cg25256723 | 1 | 169555944 |
| | cg12534424 | 7 | 127992316 |
| | cg07850604 | 14 | 36003443 |
| | cg16969368 | 17 | 57642752 |
| | cg14676592 | 16 | 49910862 |
| | cg03399905 | 15 | 79576060 |
| | cg24436906 | 2 | 242498081 |
| | cg12554573 | 3 | 51976667 |
| | cg04036898 | 1 | 46664598 |
| | cg06874016 | 17 | 40177415 |
| | cg14209784 | 10 | 88729861 |
| | cg10804656 | 10 | 22623460 |
| | cg22016779 | 2 | 230452311 |
| | cg02872426 | 6 | 110736772 |
| | cg23078123 | 1 | 68577796 |
| | cg18240400 | 10 | 46168597 |
| | cg00484358 | 1 | 110610995 |
| | cg22809047 | 2 | 101618261 |
| | cg15845821 | 19 | 16830613 |
| | cg03972838 | 22 | 39897580 |
| | cg20119148 | 19 | 18344195 |
| | cg25478614 | 3 | 187387866 |
| | cg01511567 | 11 | 57103631 |
| | cg20264732 | 16 | 68269763 |
| | cg09559780 | 16 | 50727221 |
| | cg25268718 | 14 | 24604711 |
| | cg12934382 | 3 | 51741135 |
| | cg20249566 | 19 | 16830739 |
| | cg15480367 | 14 | 93389485 |
| | cg05991454 | 4 | 147558435 |
| | cg07080372 | 11 | 796607 |
| | cg26685941 | 13 | 95952902 |
| | cg06335143 | 1 | 53308654 |
| | cg11299964 | 9 | 128469783 |
| | cg13836627 | 15 | 30113723 |
| | cg16290275 | 1 | 208042910 |
| | cg20426994 | 7 | 130418324 |
| | cg16541931 | 10 | 25463757 |
| | cg01560871 | 10 | 72545424 |
| | cg19885761 | 5 | 175223646 |
| | cg08370996 | 15 | 96874031 |
| | cg17421623 | 3 | 119187570 |
| | cg16015712 | 1 | 53308597 |
| | cg00664406 | 3 | 51740875 |
| | cg16477091 | 17 | 56833000 |
| | cg07502389 | 8 | 24771259 |
| | cg05156137 | 21 | 35898975 |
| | cg16867657 | 6 | 11044877 |
| | cg06782035 | 5 | 16179135 |

16. The method according to claim 10, wherein providing methylation levels of the plurality of methylation sites of human cells related to life style factors for the same reference population and for the subject U comprises detecting the typology of at least one SNP affecting methylation site selected from

| | | | Nucleotide | | | | | | |
|---|---|---|---|---|---|---|---|---|---|
| | | Human | number of | | | | CpG Value for allele | | |
| CpG | methyl- | chromo- | the SNP | SNP combination | | | combination | | |
| associated | QTL SNP | some # | position | #1 | #2 | #3 | #1 | #2 | #3 |
| cg00336149 | rs260500 | 19 | 58791213 | AA | AC | CC | 0.4749 | 0.4369 | 0.4165 |
| cg01511567 | rs11228995 | 11 | 57083535 | CC | CT | TT | 0.1659 | 0.1984 | 0.2362 |
| cg02872426 | rs3757351 | 6 | 110735630 | AA | AG | GG | 0.6181 | 0.5171 | 0.4167 |
| cg03224418 | rs817336 | 20 | 62603060 | CC | CG | GG | 0.4331 | 0.4198 | 0.3981 |
| cg03531211 | rs13332669 | 16 | 10998995 | AA | AT | TT | 0.6004 | 0.6128 | 0.6110 |
| cg03604011 | rs 76293297 | 8 | 18526242 | AA | AC | NA | 0.0709 | 0.0611 | NA |
| cg03972838 | rs2009774 | 22 | 39879679 | AA | AT | TT | 0.6178 | 0.6244 | 0.6409 |
| cg05156137 | rs2284576 | 21 | 35914812 | AA | AG | GG | 0.3263 | 0.3339 | 0.3539 |
| cg05329352 | rs12161672 | 10 | 112887471 | CC | CG | GG | 0.6392 | 0.5952 | 0.5659 |
| cg05923226 | rs10854136 | 19 | 15029174 | AA | AG | GG | 0.6640 | 0.6298 | 0.6027 |
| cg06121808 | rs908545 | 2 | 113389232 | AA | AG | GG | 0.4667 | 0.4182 | 0.3922 |
| cg06885782 | rs2769255 | 1 | 41245354 | CC | CT | TT | 0.5953 | 0.6113 | 0.6215 |
| cg07164639 | rs2003727 | 6 | 110738291 | CC | CT | TT | 0.3735 | 0.4473 | 0.5152 |
| cg09118625 | rs12073947 | 1 | 68363490 | AA | AC | CC | 0.6284 | 0.6395 | 0.6667 |
| cg09658497 | rs7790322 | 7 | 2830498 | CC | CT | TT | 0.7198 | 0.6457 | 0.5734 |
| cg10917602 | rs12934900 | 16 | 30923602 | AA | AT | TT | 0.6875 | 0.7014 | 0.7214 |
| cg11207515 | rs7783508 | 7 | 146897589 | CC | CT | TT | 0.4045 | 0.4332 | 0.4506 |
| cg12803068 | rs61087358 | 7 | 45017743 | AA | AG | GG | 0.6716 | 0.7344 | 0.7786 |
| cg14314729 | rs4700950 | 5 | 179825931 | AA | AG | GG | 0.4648 | 0.4606 | 0.4444 |
| cg14476101 | rs11583993 | 1 | 120255370 | AA | AG | GG | 0.5132 | 0.5968 | 0.6583 |
| cg14918082 | rs8070212 | 17 | 7829928 | AA | AG | GG | 0.8007 | 0.7821 | 0.7625 |
| cg14956327 | rs3757351 | 6 | 110735630 | AA | AG | GG | 0.3974 | 0.3478 | 0.2959 |
| cg15417641 | rs260486 | 19 | 58753937 | CC | CT | TT | 0.6909 | 0.6546 | 0.6155 |
| cg15542713 | rs2147904 | 1 | 42371414 | CC | CT | TT | 0.5759 | 0.5407 | 0.5054 |
| cg15693572 | rs9845701 | 3 | 22368388 | CC | CG | GG | 0.5217 | 0.5677 | 0.6094 |
| cg16145216 | rs2147904 | 1 | 42371414 | CC | CT | TT | 0.3968 | 0.3740 | 0.3435 |
| cg16246545 | rs11583993 | 1 | 120255370 | AA | AG | GG | 0.3837 | 0.4615 | 0.5120 |
| cg17272563 | rs186587921 | 1 | 43925902 | AC | CC | NA | 0.2039 | 0.2029 | NA |
| cg17421623 | rs62263406 | 3 | 119165162 | AA | AG | GG | 0.4530 | 0.3850 | 0.3127 |
| cg18240400 | rs76831684 | 10 | 46116032 | CC | CT | TT | 0.4968 | 0.5488 | 0.6108 |
| cg18316974 | rs115401860 | 1 | 92362377 | AA | AG | NA | 0.9328 | 0.8295 | NA |
| cg18369990 | rs4073745 | 5 | 176733341 | AA | AG | GG | 0.5838 | 0.5839 | 0.5911 |
| cg19093370 | rs146263864 | 16 | 52005011 | AG | GG | NA | 0.7788 | 0.7784 | NA |
| cg19382157 | rs4721325 | 7 | 2069846 | AA | AG | GG | 0.5160 | 0.4557 | 0.2969 |
| cg19717773 | rs7790322 | 7 | 2830498 | CC | CT | TT | 0.6719 | 0.6141 | 0.5564 |
| cg19724470 | rs10975133 | 9 | 5486390 | GG | GT | TT | 0.2080 | 0.2565 | 0.2970 |
| cg20066188 | rs727047 | 22 | 37677719 | AA | AG | GG | 0.4256 | 0.3855 | 0.3464 |
| cg20303561 | rs28651318 | 14 | 91999791 | AA | AG | GG | 0.7136 | 0.6938 | 0.6655 |
| cg21188533 | rs260452 | 19 | 58766874 | AA | AC | CC | 0.5601 | 0.6101 | 0.6573 |
| cg22132788 | rs61087358 | 7 | 45017743 | AA | AG | GG | 0.8218 | 0.8443 | 0.8867 |
| cg23480021 | rs7631738 | 3 | 22610981 | CC | CT | TT | 0.6705 | 0.6577 | 0.6829 |
| cg23681440 | rs7997900 | 13 | 27293625 | CC | CT | TT | 0.4651 | 0.4565 | 0.4396 |
| cg24049493 | rs2147904 | 1 | 42371414 | CC | CT | TT | 0.3122 | 0.2721 | 0.2360 |
| cg24172324 | rs13025087 | 2 | 232264914 | GG | GT | TT | 0.4266 | 0.3887 | 0.3583 |
| cg25212025 | rs1985313 | 10 | 34580880 | GG | GT | TT | 0.4476 | 0.4186 | 0.3876 |
| cg25809905 | rs118180322 | 17 | 42645948 | CC | CT | NA | 0.6286 | 0.6888 | NA |
| cg26337070 | rs12478164 | 2 | 85992977 | AA | AT | TT | 0.6680 | 0.6372 | 0.6159 |
| cg26394940 | rs12484609 | 22 | 46450297 | CC | CT | TT | 0.1082 | 0.1422 | 0.1847 |
| cg26614073 | rs115360749 | 3 | 46713120 | AG | GG | NA | 0.5121 | 0.5212 | NA |
| cg26718213 | rs12464946 | 2 | 242002701 | CC | CG | GG | 0.3834 | 0.3391 | 0.3158 |

and using the value under “CpG Value for allele combination” as multiplier for the associated CpG.

17. The method according to claim 10, wherein the human cells are comprised in or from solid tissue, blood, fecal or saliva sample that comprises genomic DNA.

18. The method according to claim 10, wherein the methylation value of a CpG site in a population of human cells is the average degree of methylation of said CpG methylation site in a population of at least hundreds, more preferably thousands of hundreds cells in a biological sample of the subject U.
